(19) United States
(12) Patent Application Publication
(10) Pub. No.: US 2026/0325364 A1
(43) Pub. Date: Oct. 22, 2026

(54) SPONGE FOR THE VAGINAL CANAL

(71) Applicant: UAB "AVODES", Kaunas (LT)

(72) Inventors: Audrone KACINSKAITE-SATKAUSKE, Kaunas (LT); Gabriele SAULENIENE, Kaunas (LT)

(73) Assignee: UAB "AVODES", Kaunas (LT)

(21) Appl. No.: 19/104,638

(22) PCT Filed: Sep. 19, 2023
(86) PCT No.: PCT/EP2023/075734

§ 371 Date: Feb. 18, 2025

(30) Foreign Application Priority Data
Sep. 20, 2022 (WO) PCT/EP2022/076032

(51) Int. Cl. A61F 13/20

(52) U.S. Cl. CPC A61F 13/2074

(57) ABSTRACT

This invention is directed to a sponge for the vaginal canal comprising a sponge body having a porous sponge structure. The sponge structure forms at least part of a sponge outer surface and the porous sponge structure is impregnated with a liquid composition comprising one or more bacteriocin(s). The invention also relates to a liquid composition, in particular a gel, suitable for the vaginal canal comprising one or more bacteriocin(s) in an amount which promotes the vaginal microflora by inhibiting the growth of one or more pathogen(s). The invention also relates to such a sponge or a liquid composition for use in preventing and/or treating dysbiosis, vulvovaginal candidiasis, 10 and/or bacterial vaginosis.

### FIELD OF THE INVENTION

The present invention relates to a sponge for the vaginal canal, which can be used as a tampon, wherein said sponge recovers the balance of the healthy vaginal microbiota (dominated by Lactobacilli), by selectively inhibiting pathogens (like Candida, S. aureus and others) and preserving Lactobacilli spp.

### BACKGROUND

It is estimated that around 75% of women suffer from vaginal infections such as Candidiasis at some point in life, which can be favored by the use of antibiotics, use of feminine hygiene product or douching, humid weather, taking oral contraceptives, stress and unknown causes [1]. In around 58% of cases, the condition relapses within 12 months. An estimated 8 to 10% of women are susceptible to recurrent VVC (RVVC), having 4 or more episodes per annum [1]. Most frequently this condition is caused by C. albicans, but may also result from Candida glabrata, Candida tropicalis, and Candida parapsilosis [2, 3].

Bacterial vaginosis (BV) is the most common vaginal infection (inflammation) among women of reproductive age and is associated with devastating health issues preterm births, human immunodeficiency virus, risk of sexually transmitted diseases (STD), risk of human papillomavirus [4, 5]. General population prevalence of BV is high globally, ranging from 23% to 29% each year [6]. BV-associated adverse obstetric outcomes, ranges from 8% to 51%. Bacterial vaginosis appears, when Lactobacilli disappears and microflora is overgrown by Gardnerella vaginalis and resident anaerobic vaginal bacteria [7].

Healthy women have vaginal innate defence mechanisms-vaginal microbiome dominated by Lactobacilli, regulation of cells of the immune system prevents infections by external pathogens. Several species of Lactobacilli dominate the healthy vagina, support a defense system and inhibits fungus (e.g. C. albicans) and pathogenic bacterial growth by competing for nutrients, preventing adhesion, and excreting antimicrobial compounds, like bacteriocins, bacteriocin-like substances (like H₂O₂) and others [8, 9]. Diminished dominance of Lactobacillus, cause a variety of immunological changes such as the production of pro-inflammatory cytokines/chemokines, greater recruitment of immune cells, and changes to the vaginal lining [10].

A Lactobacilli dominated microbiota is capable of expressing anti-infection protective factors inhibiting yeast/bacteria growth. Vaginal microbiota dysbiosis, when Lactobacilli spp. are outcompeted by other bacteria species, often is a cause of gynaecological diseases. In general, bacterial and fungal infections are favored when the vaginal microbiota is not in balance. Vaginal infections affect millions of women each year and these infections often cause vaginal dysbiosis—a misbalance of the vaginal microflora often causing inflammation and/or infection. Hence, the homeostasis of the microbiota is crucial and in particular imbalances in the microflora or damages to the vaginal mucosa need to be avoided. The vaginal microbiota are complex ecosystems of more than 200 bacterial species influenced for example by genes, environmental and behavioral factors [11]. Vaginal dysbiosis (or absence of Lactobacilli) and infection are even believed to account of 25-40% of preterm births. Strong deficiency of vaginal Lactobacilli are associated with serious reproductive concerns such as miscarriages and hampered in vitro fertilization (IVF) [12]. Furthermore, there is a high (30-40%) prevalence of abnormal vaginal microbiota in women undergoing IVF [13].

During menstruation, Lactobacilli are suppressed due to increased pH of the vaginal environment and other factors, resulting in 100-fold decrease in population, which can then be overgrown by pathogens [13, 14]. An increase in bacteria such as G. vaginalis is associated with menstruation, accompanied by decreased quantities of L. crispatus and other Lactobacilli were observed in healthy women with Lactobacilli dominance over the rest of the cycle [15]. In healthy women, Lactobacilli restore after menstruation unless the vaginal environment is impaired by other factors such as stress, medication, hormonal contraception, use of menstrual products. This can result in dysbiosis and lead to infections like Vulvovaginal candidiasis (VVC), Bacterial vaginosis (BV) and can even lead to pre-term birth. Medical treatment with antimycotics or antibiotics does not restore the microbiota balance and a vicious cycle of recurrent disease continues. Medical treatment is extremely limited, and the inability to prevent the frequently, symptomatic recurrences of BV to reduce serious complications such as preterm delivery, remains an acknowledged but unresolved problem [16]. In recurrent Candidiasis cases, treatment is elusive and Candida spp. evolved in highly resistant [17].

Mycotic infections (e.g. vulvovaginal Candidiasis), bacterial vaginosis, aerobic vaginosis are the most common classes. Candidiasis is an infection caused by a yeast (a type of fungus) called Candida. Candida can normally reside in the vagina without causing problems. However, Candida can multiply and cause an infection if the vaginal environment changes in a way that encourages its growth. Changes in hormone level, medicines, or changes in the immune system affect said environment.

Furthermore, one risk of using tampons is the development of toxic shock syndrome (TSS), which is why some women avoid using tampons. TSS is a rare but life-threatening disease. Up to 99% of those cases to tampon usage during menstruation. TSS is caused by toxic shock syndrome toxin 1 (TSST-1) produced by Staphylococcus aureus (S. aureus) bacteria, which can naturally form part of the vaginal microflora. However, when S. aureus multiply rapidly, TSS may develop. S. aureus growth is for example facilitated when the vaginal pH changes and when other unfavourable conditions come into play. For example, some materials of the tampon may facilitate vaginal colonization or infection more than others [18]. In particular, high absorbency can cause toxic shock syndrome, since particularly short, straight sharp fibres damage the vaginal mucosa and toxin produced by S. aureus is rapidly transferred through the body. In the United Kingdom, 40 cases of this disease are diagnosed each year. Toxic shock syndrome can progress rapidly and complications may include shock, renal failure and death.

Conventional hygienic tampons are made of synthetic materials, polymeric foam layer, cotton or rayon, that during production and bleaching emit dioxin, a health and environmentally hazardous pesticide [19]. There are cotton tampons, which usually absorb too much moisture from the vaginal mucosa (causing over-drying of the tissue). Furthermore, they are made up of small and sharp fibres that damage the vaginal mucosa and natural microflora. Such tampons can leave small cotton fragments in the vagina together with old blood particles, which can also damage the vaginal pH and cause an infection.

EP 2 448 536 A1 describes a compressed menstrual tampon comprises an elongated rod-shaped body with a filler having an insertion end and a withdrawal end having a withdrawal string. The absorbent body with the filler expands as the fluids are absorbed and can be made from viscose, cotton, or cellulose pulp. The absorbent body with the filler may also comprise or consist of an absorbent sponge. The tampon also includes means to ease the insertion, such as an applicator that conforms to the tampon shape, from which the tampon can be expelled. However, this type of menstrual tampon is rigid, absorbs excessive amounts of moisture from the vaginal mucosa, is made up of small and sharp fibres that damage the vaginal walls and natural microflora, leaving small cotton fibres with old blood particles in the vagina. It can also disturb the vaginal pH and cause infections, or may cause discomfort due to uncomfortable shape.

WO 1992/013577 A1 discloses a tampon or sanitary napkin to be used prophylactically against e.g. vaginal infections. Hence, said disclosure has the aim to prevent vaginal infections by introducing a culture of at least one lactic acid producing bacteria, such as Lactobacilli, into the tampon.

EP 3 064 072 A1 discloses a composition comprising a bacterial culture of an isolated strain of Lactobacillus is used to prevent e.g. Candidiasis. However, said disclosure is not concerned with a porous sponge structure or with the impregnation of the tampon with bacteriocins. Furthermore, when introducing Lactobacilli into a product it is assumed that the bacteria support the microflora. During menstruation, the vaginal pH-Level is, however, near neutral so that Lactobacilli are not in their preferred environment, which is acidic. Hence, Lactobacilli do not multiply under these conditions and the supportive effect for the microflora is probably marginal.

WO 2020/003113 A1 and US 2021/0121596 A1 disclose a tampon made of a porous sponge structure of naturally derived glucomannan. Said tampon is soft and avoids damage to the vaginal mucosa by being moist, soft and deformable.

### SUMMARY OF THE INVENTION

It is an object of the present invention to provide a sponge for the vaginal canal which does not have one or more of the aforementioned disadvantages. Another object of the present invention is to provide a sponge for the vaginal canal which does not harm and does not irritate the vaginal walls, does not damage the mucous membrane, reduces the likelihood of infection, facilitates insertion, prevents excessive absorption of bodily fluids, prevents leakage of menstrual fluids, and/or is convenient during use. It is yet another object of the present invention to further improve the sponge for the vaginal canal disclosed in WO 2020/003113 A1 or US 2021/0121596 A1 by further supporting the natural homeostasis of the vaginal microbiota.

One or more of the above objects is achieved by impregnating a porous sponge body with a liquid composition comprising one or more bacteriocin(s). One or more of the above objects is achieved by a liquid composition suitable for the vaginal canal comprising one or more bacteriocin(s).

This present invention provides a sponge for the vaginal canal comprising a sponge body having a porous sponge structure, as further defined in the claims. The porous sponge body forms at least part of a sponge outer surface. Furthermore, the porous sponge body is impregnated with a liquid composition comprising one or more bacteriocin(s). The present invention also provides a liquid composition suitable for the vaginal canal comprising one or more bacteriocin(s) in an amount which promotes the vaginal microflora by inhibiting the growth of one or more pathogen(s). For example, said one or more pathogens is selected from S. aureus, Candida spp, Gardnerella vaginalis and combinations thereof. Ideally, the liquid composition is an impregnant of a porous sponge.

In certain embodiments, the one or more bacteriocin(s) is/are synthesised. For example, said bacteriocins may be synthesised in a bacterial strain, such as Lactobacilli. Alternatively, bacteriocins may be synthesised by E. coli. For example, nisin may be produced by E. coli or Lactobacilli. Alternatively, said bacteriocins may be synthesised chemically. In certain embodiments, the sponge does not comprise Lactobacilli.

In certain embodiments, the sponge is a tampon. For example, the tampon may be a menstrual tampon.

In certain embodiments, the sponge body impregnated with the composition promotes the vaginal microflora during menstruation, during follicular phase, during luteal phase and/or during pregnancy.

In certain embodiments, the composition promotes the vaginal microflora, in particular by inhibiting the growth of pathogens, especially S. aureus, Candida spp, and/or Gardnerella vaginalis. Ideally, the composition promotes the vaginal microflora by selectively inhibiting the growth of pathogens. In some embodiments, the composition exhibits at least a 10-fold greater inhibition on one or more pathogen(s) than on L. gasseri and/or L. crispatus over a period of 8 hours, in particular wherein the composition exhibits at least a 10-fold greater inhibition on Gardnerella vaginalis than on L. gasseri and/or L. crispatus over a period of 8 hours, more in particular at least 50-fold, and even more in particular at least 100-fold. In other words, the vaginal microflora may be promoted by selectively inhibiting the pathogens, while inhibiting Lactobacilli at least 10-fold, at least 50 fold or at least 100-fold less. Particularly Examples 3 and 4 provide evidence that pathogens are selectively inhibited while Lactobacilli are only mildly inhibited or not inhibited.

In certain embodiments, the porous sponge structure impregnated with the composition inhibits the growth of S. aureus compared to medium alone under the same conditions. The tampon sac method may be used to test the growth of S. aureus, Candida spp, and/or Gardnerella vaginalis. Said method is for example disclosed in Nonfoux et al. (2018) [20] and as further described in Example 1 herein. In said method, the pathogenic growth is generally assessed by inserting tampons/sponges in sterile plastic bags, adding medium such as BHI broth to the bags, inoculating the medium with a pathogen, and assessing the growth of the pathogen after incubation. As positive control in said method, a bag containing inoculated medium without a tampon/sponge is generally used. As used herein, ‘medium alone’ means that the medium is inoculated with a pathogen, such as S. aureus, Candida spp and/or Gardnerella vaginalis, and that no porous sponge structure is present. Otherwise the conditions for comparing the growth of the pathogen are the same, such as the temperature of incubation, the length of incubation, the medium, and the inoculation. By performing the tampon sac method, the advantageous effect of the impregnated sponge as a whole can be assessed. This is because the combination of the sponge and the impregnation can be compared to inoculated medium. In some embodiments, the growth of S. aureus is inhibited by at least 20-fold at least 50-fold or at least 100-fold or at least 200-fold or at least 350; tested after 8 h by the tampon sac method.

In certain embodiments, the porous sponge structure impregnated with the composition inhibits the growth of Candida spp. compared to medium alone under the same conditions. In some embodiments, the growth is inhibited by at least 30% or at least 40%, or at least 50% or at least 60% or at least 70% or at least 75%; tested after 8 h by the tampon sac method.

In certain embodiments, the porous sponge structure impregnated with the composition inhibits the growth of Gardnerella vaginalis compared to medium alone under the same conditions. In some embodiments said growth of Gardnerella vaginalis is inhibited by at least 50-fold at least 100-fold or at least 250-fold or at least 500-fold or at least 1000-fold or at least 5000-fold or at least 10000-fold or at least 20000-fold; tested after 24 h by the tampon sac method.

In certain embodiments, the porous sponge structure impregnated with the composition inhibits the growth of Lactobacilli spp. compared to medium alone under the same conditions. In some embodiments, said growth of Lactobacilli spp. is inhibited by at most 12-fold; tested after 8 h by the tampon sac method.

In certain embodiments, said one or more bacteriocin(s) is/are selected from Class I, Class II, Class II, and Class IV bacteriocins from Gram-positive bacteria. In some embodiments, said one or more bacteriocin(s) is/are selected from Class I and/or Class II bacteriocin from Gram-positive bacteria.

In certain embodiments, the one or more bacteriocin(s) is/are a lantibiotic. In some embodiments, the one or more bacteriocin(s) is/are a Type A or B lantibiotic.

In certain embodiments, the one or more bacteriocin(s) is a Class II bacteriocin. In some embodiments, the Class II bacteriocin is a one-peptide bacteriocin or a two-peptide bacteriocin.

In certain embodiments, the one or more bacteriocin(s) is/are a Class IIa bacteriocin. In some embodiments, the Class IIa bacteriocin is a circular bacteriocin.

In certain embodiments, the composition further comprises lactic acid. In some embodiments, the composition comprises lactic acid in a suitable amount to provide a pH value of said composition from 3.5 to 5.5, or from 3.7 to 5, or from 3.9 to 4.5, or around 4.2. In these embodiments, the lactic acid promotes the natural Lactobacilli of the microbiota by ensuring an acidic pH.

In certain embodiments, the porous sponge structure is made of a natural or synthetic fibers. Especially, the porous sponge structure may be made out of natural fibers. In some embodiments, the porous sponge structure is made of a natural polysaccharide-based sponge material. In some embodiments, the natural polysaccharide-based sponge material is glucomannan-based sponge material. In some embodiments, the polysaccharide-based material comprises glucomannan. In some embodiment, the polysaccharide-based material is glucomannan.

In another aspect, the present invention provides a method of manufacturing a sponge for the vaginal canal as disclosed herein, the method comprising the steps of: providing a sponge for the vaginal canal comprising a sponge body having a porous sponge structure, the porous sponge structure forming at least part of a sponge outer surface; and impregnating said sponge body with a liquid composition comprising one or more bacteriocin(s) to obtain the sponge for the vaginal canal as disclosed herein.

The present invention also relates to the sponge as disclosed herein or the liquid composition as disclosed for inhibiting the growth of one or more pathogen(s) associated with a disorder or disease of the vaginal canal.

Yet another aspect of the present invention provides a sponge for the vaginal canal as disclosed herein for use in preventing or treating dysbiosis. Furthermore, the present invention relates to a sponge for use in preventing or treating vulvovaginal candidiasis, and/or bacterial vaginosis. Moreover, the present invention relates to a sponge for use in preventing toxic shock syndrome.

Furthermore, a liquid composition as described herein for use in preventing or treating dysbiosis is disclosed. Moreover, a liquid composition as described herein for use in preventing or treating vulvovaginal candidiasis, and/or bacterial vaginosis is disclosed. Additionally, a liquid composition as described herein for use in preventing toxic shock syndrome is disclosed.

In certain embodiments the liquid composition comprises a fixed amount of said one or more bacteriocin(s). A fixed amount has the advantage that there are no varying amounts of bacteriocin(s) being available in the vaginal canal. In comparison, if e.g. Lactobacilli were incorporated in the sponge, said Lactobacilli may produce a varying composition and concentration of bacteriocins. Additionally, Lactobacilli may produce a lot less bacteriocins in unfavourable conditions so that the amount of bacteriocins is ineffective. Hence, impregnating the sponge with a fixed amount of bacteriocins has the advantage that the amount of bacteriocins is always the same and thus makes the technical effect reproducible. As the amount of bacteriocins is fixed, the amount of bacteriocins is not influenced by the microenvironment.

In some embodiments, the one or more bacteriocin(s) are present at a total concentration of the liquid composition of 0.0001% (v/v) to 0.005% (v/v), 0.0002% (v/v) to 0.002% (v/v), 0.0004% (v/v) to 0.001% (v/v), 0.0005% (v/v) to 0.0014% (v/v), 0.0001% (v/v) to 0.0005% (v/v), or 0.0014% (v/v) to 0.008% (v/v).

In certain embodiment, the liquid composition is a solution. In these embodiment, the liquid composition is a liquid solution. In general, a solution is a homogenous mixture of two or more substances. In the present case, the one or more bacteriocin(s) and other possible ingredients are homogenously mixed to provide a liquid solution.

In certain embodiments, the liquid composition is a gel or a lubricant. As known in the art, a gel is a thick, clear liquid composition. As also known in the art, lubricants are known in the art for the vaginal canal and lubricants are usually water or oil-based. In some embodiment, the gel further comprises a polysaccharide such as glucomannan, a pectin, inulin or a combination thereof. Ideally, the gel further comprises glucomannan. In some embodiment, the lubricant further comprise glucomannan. In particular Example 4 provides evidence that a gel additionally comprising glucomannan even enhances the growth of Lactobacilli while selectively inhibiting pathogens. A gel or lubricant further comprising glucomannan has the additional advantage that glucomannan further improves the effect of the one or more bacteriocin(s), as glucomannan such has a prebiotic effect on the vaginal Lactobacilli. In other words, glucomannan provides ‘nutrition’ for the natural Lactobacilli and thereby natural Lactobacilli are promoted. Additionally, glucomannan helps in retaining an acidic pH. Thus, glucomannan further assist in e.g. inhibiting G. vaginalis, which are inhibited by an acidic pH. Hence, the natural microbiota is promoted and/or pathogens such a G. vaginalis are inhibited by the glucomannan. At the same time, the one or more bacteriocins specifically inhibit pathogenic bacteria and fungi, such as S. aureus and Candida. Hence, supporting the natural Lactobacilli by glucomannan and inhibiting pathogens by one or more bacteriocin(s) is simultaneously achieved in these embodiments.

A sponge for the vaginal canal according to the present invention is impregnated with a liquid composition comprising one or more bacteriocins. A liquid composition comprising said one or more bacteriocins is also disclosed. The bacteriocins are effective in promoting the vaginal microflora by inhibiting the growth of pathogens such as S. aureus, Candida albicans and/or Gardnerella vaginalis, as shown in Examples 1 and 2. At the same time, the bacteriocins are present in an amount so that the vaginal microflora is also promoted by inhibiting Lactobacilli only to a minor degree (see Example 1).

Several advantages are associated with a liquid composition comprising one or more bacteriocin(s): The vaginal mucosa is not dried, the natural vaginal pH is maintained as much as possible also during menstruation, and the supplied bacteriocins support the vaginal microflora by promoting the microflora and/or inhibiting the growth of pathogens. Furthermore, it is advantageous to supply bacteriocins as such, instead of supplying bacteria, such as Lactobacilli. This is because the production and release of bacteriocins by Lactobacilli depend on the optimal growth conditions of Lactobacilli, which require e.g. an acidic environment. During menstruation, however, the vaginal pH is near the pH of blood (pH 7) and not acidic. Various studies proved decline of Lactobacilli by a 100-fold during menstruation. While pathogenic bacteria, like Gardnerella vaginalis increased by 4-log during menstruation in healthy women [21, 23]. Hence, by impregnating the sponge with bacteriocins or by applying a liquid composition comprising one or more bacteriocin(s), the active agent is directly supplied to the vaginal microflora. Furthermore, a specified amount of bacteriocins can be supplied, whereas the amount bacteriocins produced by Lactobacilli depends strongly on the environment and thus cannot be controlled during use. The presence of bacteriocins is thus not dependent on the environmental conditions of the Lactobacilli.

The combination of the porous sponge structure and the impregnation with the liquid composition comprising one or more bacteriocin(s) is particularly advantageous. The impregnation of the porous sponge structure ensures that the bacteriocins are supplied throughout the porous sponge structure. As the porous sponge structure forms at least part of the sponge outer surface, the liquid composition comprising one or more bacteriocin(s) is in direct contract with the vaginal microflora ensuring its supportive effects. Moreover, the porous sponge structure is suitable to absorb menstrual fluids. Said porous sponge structure is suitable to prevent the occurrence of small fibres and is gentle to the vaginal walls, elastic, and shaped for an easy vaginal insertion.

Thanks to the shape and structure of the sponge, the liquid is absorbed where it is formed and safely retained inside the sponge. Since menstrual blood flows into and is retained in the pores of the sponge, the absorption pattern is fundamentally different from the conventional tampons that perform the function when the blood is absorbed by the tampon fabric itself. Menstrual blood accumulated in the pores of sponges is isolated from the vaginal walls, thus maintaining a healthier vaginal pH, where the healthy vaginal pH is more acidic (equal or less than 4.5) than the blood pH (about 7), thus preserving healthy microflora of vagina and reducing risk caused by pathogenic bacteria. This effect is further increased by the presence of one or more bacteriocins, which further inhibit the growth of pathogens and support natural Lactobacilli. The sponge structure allows the sponge to be compressed and deformed during use, ensuring convenience, but also function.

When the sponge is inserted, the sponge recovers its shape particularly fast due to the sponge structure, the material from which it is made of and its porosity. Softness, flexibility and special shape make it easy to adapt to individual physiology without causing discomfort. The sponge is designed to dynamically adopt to women's anatomy during movement so that it is comfortable to wear and effectively absorbs menstrual discharge.

### BRIEF DESCRIPTION OF THE DRAWINGS

Further objects, principles and features of the present invention may become apparent from the following description which, together with the accompanying drawings, illustrate exemplary embodiments of the present invention. However, it is to be understood that these embodiments are not intended to be limiting to the scope of the invention, but instead merely serve to illustrate exemplary implementation cases. In the drawings:

FIGS. 1a, b and c illustrate a sponge for the vaginal canal from the front, side and top without a hollow, respectively, according to one embodiment.

FIGS. 2a, b and c illustrate a sponge for the vaginal canal from the front, side, and top with a hollow on the side, respectively, according to another embodiment.

FIGS. 3a, b and c illustrate a sponge for the vaginal canal from the front, side and top with a hollow on top, respectively, according to another embodiment.

FIGS. 4a, b and c illustrate a sponge from the top, side and top with a hollow on top, respectively, according to another embodiment.

FIGS. 5a and 5b schematically illustrate insertion positions of sponges in the vaginal canal according to two different embodiments of the present invention in a vaginal canal.

FIG. 6 illustrates a sponge for the vaginal canal in perspective view according to yet another embodiment.

FIG. 7 illustrates an exemplary package containing the sponge for the vaginal canal of FIG. 6.

FIG. 8a schematically shows a sectional view of the sponge for the vaginal canal of FIG. 6, taken at a line A-A shown in FIG. 7.

FIG. 8b schematically shows a sectional view of the sponge for the vaginal canal of FIG. 6, taken at a line B-B shown in FIG. 7.

FIG. 9 provides experimental data on the inhibitory effect of an exemplary solution and gel on G. vaginalis.

FIG. 10 provides experimental data on the effect of an exemplary solution on L. gasseri.

FIG. 11a provides experimental data on the effect of an exemplary gel on L. crispatus.

FIG. 11b provides experimental data on the effect of an exemplary gel on L. crispatus and G vaginalis.

### DETAILED DESCRIPTION OF PREFERRED EMBODIMENTS

Prior to providing a detailed description of preferred embodiments of the present invention with reference to the drawings, note that identical elements are represented by the same numerals in all drawings.

It should be understood that a number of specific details are provided to reveal a complete and understandable description of exemplary embodiments of the present invention. However, it will be apparent to a person skilled in the art that the detailing of the embodiments of the invention does not limit the implementation of the invention, which may be implemented without such specific instructions. Well-known techniques, procedures and constituents have not been described in detail so that examples of implementing the invention are not misleading. Furthermore, this description should not be considered as limiting the examples presented, but only as their implementation scheme. Any equivalent feature or modification of the features of the invention is considered to fall within the scope of the invention.

In general, bacteriocins are antibacterial peptides produced by different bacteria and thereby inhibit the growth of other bacteria. Bacteriocins can also be effective against yeast. In general, bacteriocins are capable of killing bacteria or yeast. In particular, bacteriocins are generally more effective against bacteria, which do not naturally produce said bacteriocins. By specifically preserving and thereby supporting the natural Lactobacilli in the vaginal microbiota with bacteriocins, potentially pathogenic agents such as Candida albicans, S. aureus and/or Gardnerella vaginalis are inhibited from multiplying and thus vaginal infections are reduced. This may also prevent reoccurring infections. Ideally, the composition according to the present invention selectively inhibits pathogenic bacteria and fungi, such as Candida albicans, S. aureus and/or Gardnerella vaginalis, but protects the healthy microflora, such as Lactobacilli spp. In this way, the healthy vaginal microbiota balance is ideally restored and maintained. Especially, bacteriocins produced by Lactobacilli are preserving natural/vaginal Lactobacilli, but are inhibiting other bacteria and yeast, such as S. aureus, C. albicans and/or Gardnerella vaginalis.

In certain embodiments, the one or more bacteriocin(s) are synthesized. The skilled person is aware of synthesized bacteriocins, which can be commercially available or can be produced in the laboratory. Synthetized bacteriocins may be synthesised chemically or in a bacterial strain. In case said bacteriocins are synthesised in a bacterial strain, said bacteriocins may be isolated form said bacterial strain to form part of the liquid composition. For example, one or more bacteriocin may be synthesised in Lactobacilli and then isolated from said Lactobacilli. In certain embodiments, the one or more bacteriocin(s) has been produced by a bacterial strain. In case the bacteriocin(s) are chemically synthesised, the bacteriocin has not been produced by a bacterial strain. Furthermore, amino acid sequences of bacteriocins are disclosed in the art and the skilled person is aware of bacteriocin databases such as BAGEL4 (http://bagel4.molgenrug.nl/) or Bactibase (e.g. http://bactibase.hammamilab.org/main.php), which disclose the amino acids sequences of bacteriocins. Moreover, products, such as tampons, containing bacteria, e.g. Lactobacilli, may comprise naturally derived bacteriocins, wherein the bacteriocins are produced by the Lactobacilli within a product.

With regard to the liquid composition suitable for the vaginal canal, said liquid composition comprises one or more bacteriocin(s). In particular, the one or more bacteriocin(s) is the active agent of the liquid composition. As known in the art, the ‘active agent’ is capable of an activity. In some embodiments, the composition is a gel or a lubricant.

In certain embodiments of the present invention, the sponge does not comprise Lactobacilli. In some embodiments, the sponge does not comprise bacteria. In some embodiments, the liquid composition does not comprise bacteria such as Lactobacilli. In certain embodiments, the sponge body impregnated with the composition promotes the vaginal microflora at any stage of the menstrual cycle and/or during pregnancy. Similarly, the liquid composition promotes the vaginal microflora at any stage of the menstrual cycle and/or during pregnancy in some embodiments. Specifically, the sponge body impregnated with the composition or the composition may promote the vaginal microflora during menstruation, during follicular phase, during luteal phase and/or during pregnancy. Using the sponge during menstruation has the particular advantage that the menstrual blood is retained within the pores of the sponge without drying the vaginal mucosa while providing bacteriocins to the natural Lactobacilli of the vaginal canal and thereby inhibit potential pathogens, such as S. aureus, Candida albicans and/or Gardnerella vaginalis. During follicular phase and luteal phase, the sponge body impregnated with the composition or the liquid composition provide the particular advantage of providing one or more bacteriocin(s) to the vaginal microflora. Particularly recurring vaginal infections, such as vulvovaginal candidiasis or bacterial vaginosis, may be prevented or treated during follicular phase and luteal phase. As the sponge is impregnated with a liquid composition comprising the one or more bacteriocin(s), the sponge is moist and does not dry the vaginal mucosa. Particularly during pregnancy, vaginal infections are recurring. Hence, the sponge, compared to conventional tampons, provides the particular advantage of promoting the natural vaginal microbiota with the bacteriocins while not drying the mucosa.

In certain embodiments, the sponge is a tampon. In some embodiments, the tampon is a menstrual tampon.

According to the present invention, the sponge body is impregnated with a liquid composition comprising one or more bacteriocins. As used herein, ‘impregnation’ means that the sponge body is soaked with a liquid composition so that the liquid composition is distributed in the sponge body. For example, the sponge can be made out of fibers, such as polysaccharide fibres. In this case, the polysaccharide fibers would be impregnated with the liquid composition. If in use, the pores of the porous sponge structure can absorb menstrual fluids, which is advantageous as the sponge does not dry the vaginal mucosa by retaining the menstrual fluid in the pores.

In certain embodiments, the composition promotes the vaginal microflora by inhibiting the growth of one or more pathogens. In some embodiments, the sponge body impregnated with the composition promotes the vaginal microflora by inhibiting the growth of one or more pathogens. By promoting the vaginal microflora, the composition or the sponge body impregnated with the composition positively influences the microflora. For example, the microflora is positively influenced by promoting the health of the microflora. For instance, the microflora is positively influenced by promoting Lactobacilli spp. Ideally, the composition or the sponge body impregnated with the composition promote a balanced vaginal microflora. A balanced vaginal microflora is Lactobacilli spp. dominated. Ideally, the composition or the sponge body impregnated with the composition promotes the dominance of Lactobacilli spp. in the vaginal microflora. Lactobacilli spp. is dominant when the genus Lactobacilli spp. is most abundant in the microflora. In turn, no other genus is more abundant than Lactobacilli spp. in the microflora when Lactobacilli spp. is dominant. Ideally, the health of the microflora is promoted by the composition or the sponge body impregnated with the composition. Specifically, the health of the microflora may be promoted by promoting the dominance of Lactobacilli spp. By inhibiting the growth of pathogens, the metabolic activity may be shifted. For example, the composition or the sponge body impregnated with the composition inhibits the growth of S. aureus, Candida spp, and/or Gardnerella vaginalis. Examples 1 and 2 herein provides data on the inhibition of the growth of S. aureus, Candida spp, and Gardnerella vaginalis specifically. Advantageously, the composition or the sponge body impregnated with the composition promote the vaginal microflora so that a possible disbalance of the vaginal microflora, e.g. during vulvovaginal candidiasis, bacterial vaginosis, may be overcome. Further advantageously, the composition or the sponge body impregnated with the composition promote the vaginal microflora so that a Lactobacilli spp. dominated microflora may be obtained or maintained.

In certain embodiments, the porous sponge structure impregnated with the composition inhibits the growth of S. aureus compared to medium alone under the same conditions, wherein the growth is inhibited, for example, by at least 20-fold at least 50-fold or at least 100-fold or at least 200-fold or at least 350-fold; tested after 8 h by the tampon sac method. The skilled person is aware of the tampon sac method. Said method has been described e.g. in Nonfoux L, et al. (2018) [7]. In addition, said method was also used in Example 1 herein and further detail is provided. Based on the data disclosed in Example 1, the skilled person is able to test suitable substances and assess the inhibition of a pathogen such as S. aureus, Candida spp and/or Gardnerella vaginalis. Similarly, in some embodiments, the liquid composition inhibits the growth of S. aureus compared to medium alone under the same conditions, wherein the growth is inhibited, for example, by at least 20-fold at least 50-fold or at least 100-fold or at least 200-fold or at least 350-fold.

In some embodiments, the porous sponge structure impregnated with the composition inhibits the growth of S. aureus compared to medium alone under the same conditions, wherein the growth is inhibited by at least 100-fold; tested after 8 h by the tampon sac method. In some embodiments, the porous sponge structure impregnated with the composition inhibits the growth of S. aureus compared to medium alone under the same conditions, wherein the growth is inhibited by at least 200-fold; tested after 8 h by the tampon sac method. In some embodiments, the porous sponge structure impregnated with the composition inhibits the growth of S. aureus compared to medium alone under the same conditions, wherein the growth is inhibited by at least 350-fold; tested after 8 h by the tampon sac method.

In certain embodiments, the porous sponge structure impregnated with the composition inhibits the growth of Candida spp. compared to medium alone under the same conditions, wherein the growth is inhibited, for example, by at least 30% or at least 40%, or at least 50% or at least 60% or at least 70% or at least 75%; tested after 8 h by the tampon sac method. Similarly, in some embodiments, the liquid composition inhibits the growth of Candida spp. compared to medium alone under the same conditions, wherein the growth is inhibited, for example, by at least 30% or at least 40%, or at least 50% or at least 60% or at least 70% or at least 75%. In addition, the data of the Examples provide further detail and demonstrates such an inhibition of growth. In some embodiments, the porous sponge structure impregnated with the composition inhibits the growth of Candida spp. compared to medium alone under the same conditions, wherein the growth is inhibited by at least 60%; tested after 8 h by the tampon sac method. In some embodiments, the porous sponge structure impregnated with the composition inhibits the growth of Candida spp, compared to medium alone under the same conditions, wherein the growth is inhibited by at least 70%; tested after 8 h by the tampon sac method. In some embodiments, the porous sponge structure impregnated with the composition inhibits the growth of Candida spp. compared to medium alone under the same conditions, wherein the growth is inhibited by at least 80%; tested after 8 h by the tampon sac method. In certain embodiments, the porous sponge structure impregnated with the composition inhibits the growth of Candida spp. compared to medium alone under the same conditions, wherein the growth is, for example, inhibited by at least 50%, or at least 60%, or at least 70%, or at least 80% and or at least 85%; tested after 48 h by the tampon sac method.

In certain embodiments, the porous sponge structure impregnated with the composition inhibits the growth of Gardnerella vaginalis by at least 50-fold at least 100-fold or at least 250-fold or at least 500-fold; tested after 24 h by the tampon sac method. Similarly, in some embodiments, the liquid composition inhibits the growth of Candida spp. compared to medium alone under the same conditions, wherein the growth is inhibited, for example, by at least 30% or at least 40%, or at least 50% or at least 60% or at least 70% or at least 75%. In addition, the data of the Examples provide further detail and demonstrates such an inhibition of growth. In some embodiments, the porous sponge structure impregnated with the composition inhibits the growth of Gardnerella vaginalis at least 1000-fold tested after 24 h by the tampon sac method. In some embodiments, the porous sponge structure impregnated with the composition inhibits the growth of Gardnerella vaginalis at least 5000-fold tested after 24 h by the tampon sac method. In some embodiments, the porous sponge structure impregnated with the composition inhibits the growth of Gardnerella vaginalis at least 10000-fold tested after 24 h by the tampon sac method. In some embodiments, the porous sponge structure impregnated with the composition inhibits the growth of Gardnerella vaginalis at least 20000-fold tested after 24 h by the tampon sac method.

In certain embodiments, the porous sponge structure impregnated with the composition inhibits the growth of Lactobacilli spp. compared to medium alone under the same conditions by at most 12-fold; tested after 8 h by the tampon sac method. An inhibition by at most 12-fold corresponds to a inhibition by at most 1.079 log₁₀. In some embodiments, the porous sponge structure impregnated with the composition inhibits the growth of Lactobacilli spp. compared to medium alone under the same conditions by at most 20-fold, at most 15-fold, at most 14-fold or at most 13-fold; tested after 8 h by the tampon sac method. Similarly, in some embodiments, the liquid composition inhibits the growth of Lactobacilli spp. compared to medium alone under the same conditions by at most 12-fold.

In certain embodiments, the one or more bacteriocin(s) is/are a bacteriocin from Gram-positive and/or Gram negative bacteria. In some embodiments, the one or more bacteriocin(s) is/are a bacteriocin from Gram-positive bacteria.

In some embodiments, the one or more bacteriocin(s) is/are selected from Class I, Class II, Class II, and Class IV bacteriocins from Gram-positive bacteria. For example, the one or more bacteriocin(s) may be selected from Class I and/or Class II bacteriocin from Gram-positive bacteria.

In certain embodiments, the one or more bacteriocin(s) is/are a bacteriocin from Lactococcus lactis and/or Lactobacillus. Furthermore, the one or more bacteriocin(s) may be from the Lactobacillies order. Specifically, Lactobacillies order comprises Lacto bacillus, Leuconostoc, Pediococcus, Lactococcus, Streptococcus, Aerococcus, Carnobacterium, Enterococcus, Oenococcus, Tetragenococcus, Vagococcus, an Weissella. Alternatively, the one or more bacteriocin(s) may be from a bacteriocin Sporolactobacillus. Bacteriocins originating from Lactobacilli have the particular advantage that said bacteriocins have little inhibiting effect on Lactobacilli but a large effect on other bacteria. For example, the one or more bacteriocin(s) may be a bacteriocin from Lactobacillus crispatus and/or Lactobacillus gasseri. In some embodiments, the one or more bacteriocin(s) is/are a lantibiotic. Lantibiotics are a class of polycyclic peptide antibiotics that contain the characteristic thioether amino acids lanthionine or methyllanthionine, as well as the unsaturated amino acids dehydroalanine, and 2-aminoisobutyric acid. Lantibiotics are produced by a Gram-positive bacteria to attack other Gram-positive bacteria such as S. aureus.

In particular, the one or more bacteriocin(s) may be a Type A or B lantibiotic. For example, the lantibiotic may be selected from nisin, lactosin, lacticin, carnocin, cytolysin, subtilin, gallidermin, epidermin, mersacidin, actagardine, cinnamycin, duramycin, sublancin and plantaricin. In certain embodiments, the lantibiotic is nisin. In some embodiments the one or more bacteriocin(s) comprise(s) nisin. In embodiments in which the one or more bacteriocin(s) is/are nisin, the nisin may be selected from nisin A, nisin Z, nisin U, nisin Q, and nisin F. In some embodiments, the nisin is selected from nisin A, nisin Z or a combination thereof. Ideally, the nisin is nisin Z. Alternatively, the nisin is nisin A. In some embodiment, the composition comprises one bacteriocin. Said one bacteriocin may be nisin, e.g. nisin A or nisin Z. Ideally, said composition has an acidic pH, such as a pH from 3.5 to 5.5. In certain embodiments, the composition comprises one bacteriocin being nisin and lactic acid in an amount ensuring a pH value of said composition from 3.5 to 5.5, or from 3.7 to 5, or from 3.9 to 4.5, or around 4.2.

In certain embodiments, the one or more bacteriocin(s) is a Class II bacteriocin. In these embodiments, the Class II bacteriocin may be a one-peptide bacteriocin or a two-peptide bacteriocin. In some embodiments the Class II bacteriocin is a one-peptide bacteriocin. In embodiments in which the one or more bacteriocin is a one-peptide bacteriocin, said one-peptide bacteriocin may be selected from a pediocin-like bacteriocin and/or a nonpediocin-like bacteriocin. In some embodiments, the one-peptide bacteriocion is a nonpediocin-like bacteriocin.

In certain embodiments, the one or more bacteriocin(s) is/are selected from pediocin PA1, leucocin A, sakacin P, curvacin A, mesentericin Y105, carnobacteriocin BM1, carnobacteriocin B2, enterocin A, pikscicolin 126, bavaricin MN, piscicocin V1a, lactococcin A, lactococcin B, divergicin 750, lactococcin 972, enterocin B, carnobacteriocin A and crispacin A. In some embodiments, the one or more bacteriocin(s) is/are selected from lactococcin A, lactococcin B, divergicin 750, lactococcin 972, enterocin B, carnobacteriocin A and crispacin A. In some embodiments, the one or more bacteriocin(s) is/are crispacin A.

In certain embodiments, the one or more bacteriocin(s) is/are a Class IIa bacteriocin, for example, a circular bacteriocin. In some embodiments, the circular bacterioncin is selected from gassericin A, reutericin A, butyrivibricin A, lactocyclicin Q, circularin A, leucocyclicin Q, amylocyclin A, carnocyclin A, uberolysin A, AS-48, and garvicin ML, preferably wherein the circular bacteriocin is gassericin A.

In certain embodiments, the composition comprises one, two or three bacteriocin(s). In some embodiments, the composition comprises one or two bacteriocin(s). For example, the composition may comprise one Class I and one Class II bacteriocin. An exemplary Class I bacteriocin may be nisin and an exemplary Class II bacteriocin may be a one-peptide bacteriocin, such as crispacin A. In another example, the composition may comprise one Class I and one circular bacteriocin. An exemplary Class I bacteriocin may be nisin and an exemplary circular bacteriocin may be gassericin A.

In certain embodiments, the composition may have a pH value from 3.5 to 5.5. In certain embodiments, the composition may have a pH value from 3.7 to 5, the composition may have a pH value from 3.9 to 4.5, the composition may have a pH value at around 4.2. In certain embodiments, the composition further comprises lactic acid. For example, the composition may comprise lactic acid in a suitable amount to provide a pH value of said composition from 3.5 to 5.5. The composition may also comprise lactic acid in a suitable amount to provide a pH value of said composition from 3.7 to 5. Alternatively, the composition may comprise lactic acid in a suitable amount to provide a pH value of said composition from 3.9 to 4.5. In some embodiments, the composition may comprise lactic acid in a suitable amount to provide a pH value of said composition of about 4.2. The lactic acid may be synthesised or may stem from natural sources such as lactic acid bacteria.

In certain embodiments, the skilled person is able to determine a suitable concentration of bacteriocin(s) by using the tampon sac method as disclosed in Example 1 and 2. In certain embodiments, the one or more bacteriocin(s) are present at a total concentration of the liquid composition from 0.000001% (v/v) to 5% (v/v). Alternatively, the total concentration of said one or more bacteriocin(s) may be present in the composition from 0.00001% to 2% of the liquid composition. The total concentration of said one or more bacteriocin(s) may also be present in the composition from 0.00005% to 1% of the liquid composition. For example, the total concentration of said one or more bacteriocin(s) may be present in the composition from 0.0001% to 0.5% of the liquid composition. Alternatively, the total concentration of said one or more bacteriocin(s), such as crispacin A, may be present in the composition from 0.0005% to 0.25% of the liquid composition. In particular, the one or more bacteriocin(s) may be present at a total concentration of the liquid composition from 0.0001% (v/v) to 0.005% (v/v). Ideally, the one or more bacteriocin(s), such as gassericin A, may be present at a total concentration of the liquid composition from 0.0002% (v/v) to 0.0020% (v/v). In some embodiments, the one or more bacteriocin(s), such as nisin Z, are present at a total concentration of the liquid composition from 0.0005% (v/v) to 0.0014% (v/v). In some embodiments, the one or more bacteriocin(s), such as nisin A, are present at a total concentration of the liquid composition from 0.0005% (v/v) to 0.0014% (v/v). In certain embodiments, the one or more bacteriocin(s), such as nisin A and/or Z, are present at a total concentration of the liquid composition from 0.0006% (v/v) to 0.0013% (v/v). In certain embodiments, the one or more bacteriocin(s), such as nisin A and/or Z, are present at a total concentration of the liquid composition from 0.0007% (v/v) to 0.0012% (v/v). In certain embodiments, the one or more bacteriocin(s), such as nisin A and/or Z, are present at a total concentration of the liquid composition from 0.0008% (v/v) to 0.0011% (v/v). In certain embodiments, the one or more bacteriocin(s), such as nisin A and/or Z, are present at a total concentration of the liquid composition from 0.0009% (v/v) to 0.0010% (v/v). Alternatively, the one or more bacteriocin(s), such as curvacin A, may be present at a total concentration of the liquid composition from 0.0001% (v/v) to 0.0005% (v/v). Alternatively, the one or more bacteriocin(s), such as lacticin, may be present at a total concentration of the liquid composition from 0.0014% (v/v) to 0.008% (v/v).

In certain embodiments, the sponge is moist. In these embodiments, the sponge is slightly wet and not dry. The sponge is impregnated with a liquid composition and thus moist. Said moist sponge is soft and gentle to vaginal epithelial cells of the mucous membrane, which keeps the mucosal lining moist and healthy. By being soft and thereby easily deformable, the sponge is also comfortable to use.

The composition may further comprise one or more excipients. Exemplary excipients are benzalkonium chloride, benzethonium chloride, benzoic acid, benzyl alcohol, boric acid, sodium propionate, sorbic acid, propylene glycol, potassium lactate, calcium acetate, calcium chloride, sodium sorbate, sodium benzoate, sodium chloride, sodium formate, sodium propionate, ethylenediaminetetraacetic acid (EDTA), sodium lactate, organic and inorganic salts and acids, like sorbic acid, ascorbic acid, propionic acid, and/or fumaric acid. These excipients preserve the bacteriocin(s) in the composition. These excipients also preserve the sponge. Excipients thus have the advantage that the bacteriocins are stabilized and the sponge material is preserved.

The skilled person is also aware of suitable buffering solutions for the composition. For example, the composition may further comprise sodium chloride and EDTA. Alternatively, the buffer solution may also comprise GPB (Gelatin Phosphate Buffer), sodium phosphate buffer, Tris-glycine and Tris-tricine, octanol phosphate, sodium citrate combined with sodium diacetate (BSCSD), EDTA, sodium sulphate, sodium phosphate, sodium acetate and sodium citrate, EGTA (ethylene glycol-bis(β-aminoethyl ether). The composition may also comprise benzalkonium chloride, benzethonium chloride, benzoic acid, benzyl alcohol, boric acid, sodium propionate, sorbic acid, propylene glycol, potassium lactate, calcium acetate, and/or calcium chloride. For example, the composition may also comprise the following components at a concentration of 0.01-0.02% v/v benzalkonium chloride, 0.01-0.02% v/v benzethonium chloride, 0.1-0.2% v/v benzoic acid, 5-10% v/v sodium propionate, and/or 0.05-0.2% v/v sorbic acid.

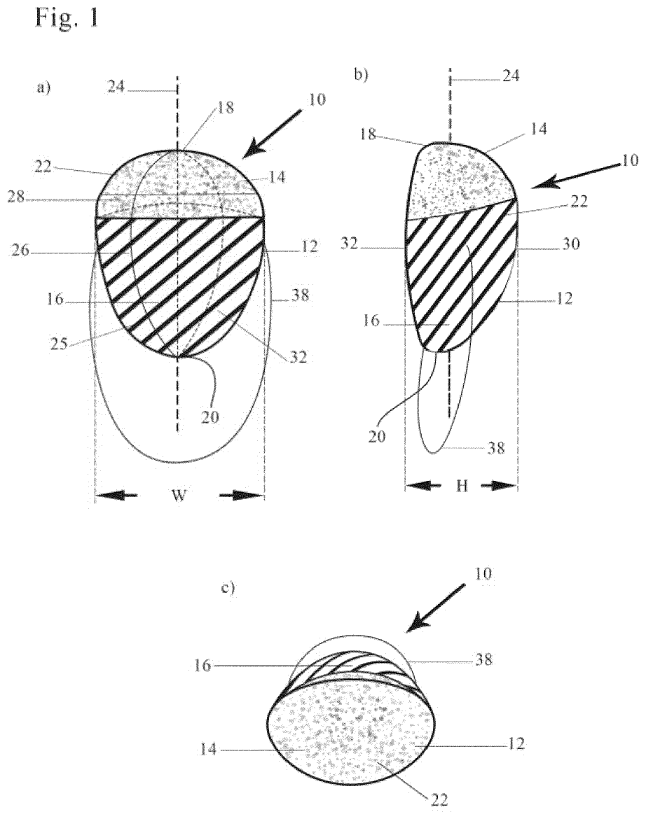

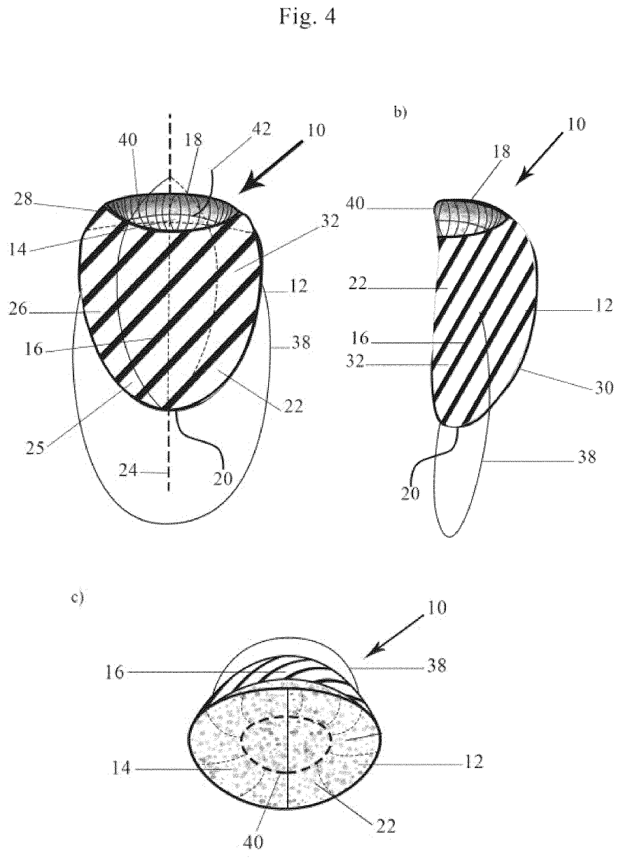

Exemplary embodiments of a sponge for the vaginal canal 10, as depicted in FIGS. 1 to 4, include an absorbent sponge body 12 comprising a porous sponge structure 14. The material of the sponge structure 14 may be a plant-derived material that can easily deform and readily reform to a non-deformed, or partially deformed state. Such material can be produced from Amorphophallus konjac (Konjac plant) plant root powder by production of glucomannan and subsequently processing it by a specific method in order to obtain the sponge structure, or it can be made from other naturally occurring polysaccharides, such as starch, cellulose, chitosan, chitin, and their mixtures, without limitation.

In certain embodiments, the porous sponge structure is made of a natural polysaccharide-based sponge material. Said natural polysaccharide-based sponge material may be glucomannan-based sponge material. In certain embodiments, the sponge is made out of completely natural-occurring materials, such as glucomannan. Due to its biological origin, glucomannan-based sponge and all its additives are biodegradable and decompose naturally in nature. In these embodiments, the sponge fully biodegradable thus friendly to the environment. In these embodiments, the sponge biodegrades within 30-90 days in natural environment (depending on environment, the moisture level and microorganisms quantity, species). Glucomannan further improves the effect of the one or more bacteriocin(s), as Glucomannan such has a prebiotic effect on the vaginal Lactobacilli. In other words, glucomannan provides ‘nutrition’ for the natural Lactobacilli and thereby natural Lactobacilli are promoted. Additionally, glucomannan helps in retaining an acidic pH. Thus, glucomannan further assist in e.g. inhibiting G. vaginalis, which are inhibited by an acidic pH. Hence, the natural microbiota is promoted and/or pathogens such a G. vaginalis are inhibited by the glucomannan. At the same time, the one or more bacteriocins specifically inhibit pathogenic bacteria and fungi, such as S. aureus and Candida. Hence, supporting the natural Lactobacilli by glucomannan and inhibiting pathogens by one or more bacteriocin(s) is simultaneously achieved in these embodiments.

In general, glucomannan is a substance used in food industry, also in medicine for wound dressing which, together with vaginal Lactobacteria, inhibits the growth of bacteria such as Staphylococcus aureus and S. typhimurium. The sponge may not contain toxic chemicals and the resulting final product may be biocompatible with the human body, i.e. ensures protection of natural vaginal microflora and healthy intimate hygiene. Glucomannan is hydrophilic and, thanks to its unique molecular structure, attracts water molecules, so the sponge walls are always moist and come in contact with the vaginal walls with a molecular barrier of liquid-protecting extremely sensitive vaginal walls.

In certain embodiments, the porous sponge structure makes up for at least 70% or at least 80% or at least 90% or at least 95% or at least 97% or at least 99% by volume of the sponge body. In particular, the porous sponge structure may make up the entire sponge body. The sponge body may be entirely made up from glucomannan.

According to the present invention, the porous sponge structure forms at least part of a sponge outer surface. This ensures that the impregnation with the liquid composition comprising the one or more bacteriocins is in direct contact with the vaginal microflora. This has the advantage that the one or more bacteriocin(s) can support the natural Lactobacilli by inhibiting potential pathogens such as S. aureus and/or Candida. In some embodiments, the sponge structure forms at least 30%, or at least 40% or at least 50% or at least 60%, or at least 70% or at least 80% or at least 90% of the sponge outer surface. In some embodiment, the porous sponge structure forms 100% of the sponge outer surface. This embodiment is particularly advantageous, as the surface of the sponge and the vaginal microflora are in maximal contact.

In certain embodiments, the sponge comprises the sponge body having an insertion end and a withdrawal end. In some embodiments, the sponge comprises a removal facilitator for facilitating removal of the sponge body from a human vaginal canal, the removal facilitator connected to the sponge body and extending from the withdrawal end of the sponge body. Additionally, the sponge may be placed into the vaginal canal by an applicator.

In some embodiments, the sponge structure is exposed at least in a region of the body outer surface that is located at or near the insertion end of the sponge body. In some embodiments, the body outer surface has a fluid capture depression formed at least partially by an exposed portion of the sponge structure. In these embodiments, the fluid capture depression may be located closer to the insertion end than to the withdrawal end of the sponge body.

In certain embodiments, the sponge body comprises a fluid impervious layer covering the sponge structure in a partial area of the body outer surface. In these embodiments, the fluid impervious layer may be made of a non-porous, glucomannan-based material.

In the embodiments of FIGS. 1a to 4c, the sponge body 12 also includes a liquid impermeable layer 16 (i.e. fluid impervious layer; hatched in the drawings) which at least partially covers the sponge structure 14 to retain the moisture in the sponge body 12 until its use and prevent leakage of liquid from the sponge body 12 during its use. The liquid impermeable layer 16 covers in certain embodiments more than half, half or less than half of the outer surface of the sponge structure 14.

The sponge also may have an outer fluid-impervious layer that provides fluid retention in the sponge even when its deformed during use, i.e. prevents leakage of fluid as the person moves causing squeezing, bending and similar deformation of the sponge material.

In certain embodiments, the liquid impermeable layer (i.e. fluid impervious layer) 16 is a non-absorbent, non-porous layer having a thickness, e.g., from 0.01 mm to 2 mm. For example, the liquid impermeable layer may be 0.01 mm. It is to be understood that the indicated thickness range of the liquid impermeable layer 16 is merely exemplary and not intended to be limiting to the scope of the invention. For example, the liquid impermeable layer 16 may have in other embodiments a thickness of only a few tenths of a millimeter. The liquid impermeable layer 16 may be made of a glucomannan-based material. It can be directly formed on the sponge structure 14 after the sponge structure 14 has been produced. Other polysaccharides than glucomannan (e.g., agar, xanthan gum, carrageenan or starch) can be used in alternate embodiments as a basis for the production of the liquid impermeable layer 16. It is to be understood, however, that the invention is not limited to the use of a polysaccharide material for the liquid impermeable layer 16 and that a latex or similar polymer material or a resin material can be used instead.

In embodiments which omit the liquid impermeable layer 16, the sponge body 12 may be formed entirely by the sponge structure 14. The sponge structure 14 may thus make up for up to 100% by volume of the sponge body 12. In embodiments which have the liquid impermeable layer 16, the sponge structure 14 may make up for, e.g., 90% or more by volume of the sponge body 12. The remainder of the volume of the sponge body 12 may be formed by the liquid impermeable layer 16. In certain embodiments, the sponge structure 14 makes up for at least 95% or at least 97% by volume of the sponge body 12.

The sponge also may include an applicator for easy insertion and a cord, string or ring for convenient removal, but it can also be used without an applicator.

In certain embodiments, the sponge body 12 has an insertion end 18, a withdrawal end 20 and a body outer surface 22. The insertion end is the leading end of the sponge body as the sponge body is shoved into a human vaginal canal, the withdrawal end is the trailing end of the sponge body during insertion of the sponge body. The body outer surface 22 is defined by the outer surface of the sponge body 12. Where the sponge structure 14 has one or more exposed portions (i.e. exposed to the outside of the sponge body 12), these exposed portions form at least part of the body outer surface 22. Where the liquid impermeable layer 16 is present and covers at least part of the sponge structure 14, the liquid impermeable layer 16 forms at least part of the body outer surface 22. The extension of the sponge body 12 from the insertion end 18 to the withdrawal end 20 defines a longitudinal extension (or direction) of the sponge body 12. The longitudinal extension is illustrated by a dashed line 24 in some of the Figures.

In certain embodiments, the sponge body has a length dimension in a direction from the withdrawal end to the insertion end, wherein the fluid impervious layer covers the sponge structure continuously in a region of the body outer surface that extends from the withdrawal end over at least one fourth or at least one third or at least one half of the length of the sponge body.

The sponge body 12 may have an insertion end 18, a withdrawal end 20 and a body outer surface 22. The insertion end is the leading end of the sponge body as the sponge body is shoved into a human vaginal canal, the withdrawal end is the trailing end of the sponge body during insertion of the sponge body. The body outer surface 22 is defined by the outer surface of the sponge body 12. Where the sponge structure 14 has one or more exposed portions (i.e. exposed to the outside of the sponge body 12), these exposed portions form at least part of the body outer surface 22. Where the liquid impermeable layer 16 is present and covers at least part of the sponge structure 14, the liquid impermeable layer 16 forms at least part of the body outer surface 22. The extension of the sponge body 12 from the insertion end 18 to the withdrawal end 20 defines a longitudinal extension (or direction) of the sponge body 12. The longitudinal extension is illustrated by a dashed line 24 in some of the Figures.

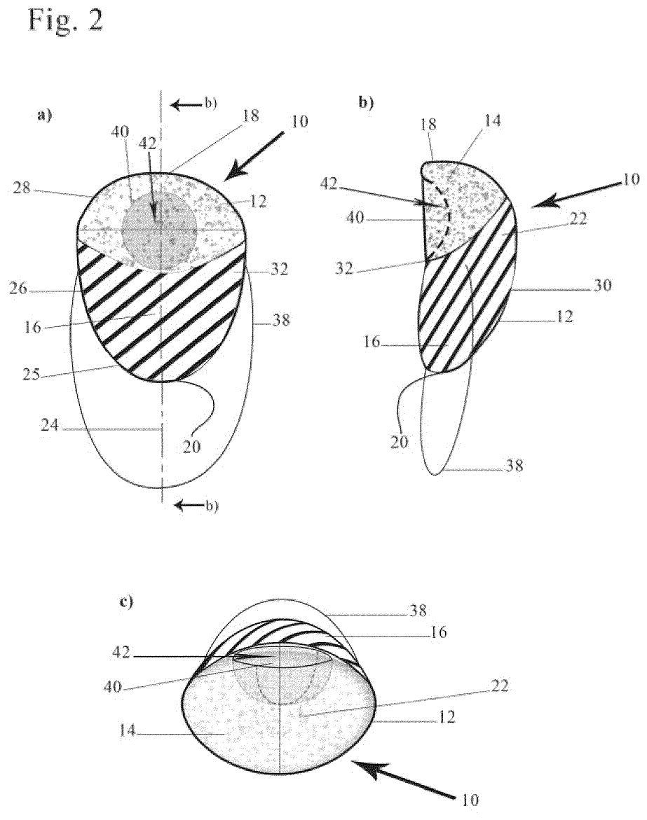

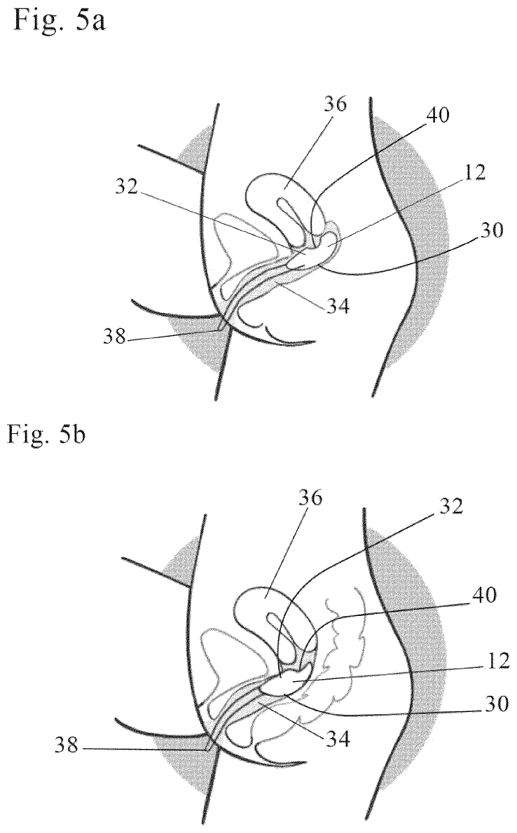

As seen in the longitudinal direction 24 from the withdrawal end 20 to the insertion end 18, the sponge body 12 has a lower portion 25, a central portion 26 and an upper portion 28, in that order. The sponge body has opposite main faces 30, 32 each extending between the withdrawal and insertion ends 18, 20. The main face 30 is a rear face of the sponge body 12 and the main face 32 is a front face, considering an intended, proper insertion position of the sponge body 12 in a human vagina 34, in which insertion position the main face 30 faces substantially downward and away from a uterus 36 of the wearer and the main face 32 faces substantially upward and toward the uterus 36, as shown in FIGS. 5a and 5b. In the embodiments shown in FIGS. 1a to 5b, the main face 30 has convexity in the longitudinal direction 24 over essentially the entire longitudinal length of the sponge body 12. The main face 32 is generally formed with a smaller degree of convexity in the longitudinal direction 24 than the main face 30. In some embodiments (see, e.g., FIGS. 2b, 3b, 4b, 5a, 5b), the main face 32, when viewed in the longitudinal direction 24, may be formed with substantially no curvation or with concavity in at least a part of the longitudinal length of the sponge body 12.

When viewed in cross-sectional profile (i.e. in a transverse plane), the main face 30 has convexity, with the main face 32 generally having a smaller degree of convexity than the main face 30 or no convexity at all. The situation of no convexity covers both a substantially straight extension of the main face 32 in the transverse plane or a profile of the main face 32 having one or more concavely shaped portions. Viewed in a cross-section orthogonal to the longitudinal direction 24, the sponge body 12 thus has an irregular, rotationally asymmetrical shape. This irregular cross-sectional shape of the sponge body may extend continuously over a major part of the longitudinal length of the sponge body 12, e.g., more than 50% or more than 60% or more than 70% or more than 80% or more than 90% of the length of the sponge body 12. Certain embodiments, the sponge body 12 may also comprise an outer reinforcing casing (not shown in the drawing), which reinforces the structure of the sponge body 12 and helps maintain the desired shape.

Exemplary approximate sizes of the sponge body 12 may be as follows:
Size 1: length 4.0-4.5 cm; width 1.5-2.0 cm; thickness ca. 1.0 cm
Size 2: length 4.5 cm; width 3.6 cm; thickness 1.9 cm
Size 3: length 5.8 cm; width 4.5 cm; thickness 2.3 cm
Size 4: length 6.0 cm; width 3.9 cm; thickness 2.2 cm

In some embodiments, the sponge 10 comprises a removal facilitator 38, such as a thread, ring or string. The removal facilitator 38 is coupled to the sponge body 12 and extends from the withdrawal end 20 in a manner conventionally known per se. The removal facilitator 38 may be attached at two ends to the sponge body 12 approximately in the central portion 26 or the upper portion 28 or the lower portion 25, on opposite sides of the sponge body 12 so as to form a U-shaped loop that extends from one lateral side of the sponge body 12 to the other and forms the possibility of a gap between at least the lower portion 25 of the sponge body 12 and a gripping part of the removal facilitator 38. The removal facilitator 38 can also be formed as a single element that is pierced through the sponge body 12 at any location, such as the upper, central or lower portions 28, 26, 25 of the sponge body 12.

In some embodiments, the sponge body 12 is irregular in shape, similar to an oval, or any other sleek shape. The sponge body 12 may be generally wider near its insertion end 18 then near its withdrawal end 20. The mentioned flattened profile, according to which the width of the sponge body 12 (designated W in FIG. 1a) is larger than its height, or thickness (designated H in FIG. 1b) over at least a major part of the longitudinal length of the sponge body 12, serves to reproduce the natural biological vaginal canal form (https://i.pinimg.com/originals/d9/d1/3 b/d9d13b90a7d2c5ba6e5314f2c8b3258f.jpg) to create a comfortable experience for the user. This is related to the fact that the use of solid form devices that create pressure to the front and back of the vagina (where many nerve endings are centered) generates additional pain or discomfort for the user. As can be seen from FIGS. 1a, 2a, 3a, 4a, the upper portion 28 is generally formed with a larger width than the lower portion 25. When inserted in the vagina, the sponge body 12 is located with its upper portion 28 closer to the origin of the menstrual fluids than with its lower portion 25 (cf. FIGS. 5a, 5b). The greater width of the upper portion 28 can thus enhance the absorption of the menstrual fluids in the sponge structure 14, whereas the lower portion 28 can efficiently retain the accumulated fluids in spite of its reduced width whilst ensuring high wearing comfort.

In the embodiments of FIGS. 2a to 5b, the sponge body 12 is formed with a hollow (recessed portion) 40 exhibiting a generally concavely shaped absorption surface 42 at which the sponge structure 14 is exposed to the outside of the sponge body 12. If present, the liquid impermeable layer 16 does not cover the sponge structure 14 at the absorption surface 42. The hollow 40 may also be referred to as a fluid capture depression or pocket. The surface area of the absorption surface 42 is in certain embodiments at least 1.5 cm² or at least 2.0 cm². The hollow 40 may be concavely shaped in all directions, resembling, e.g., a bowl shape or the like.

In certain embodiments of the invention, the liquid impermeable layer 16 may cover the entire body outer surface 22 of the sponge body 12 up to the perimetrical edge of said hollow 40. In other embodiments of the invention, the liquid impermeable layer 16 may wrap the sponge structure 14 only up to a distance from the perimetrical edge of the hollow 40, so that the sponge structure 14 remains uncoated by the liquid impermeable layer 16 (i.e. exposed to the outside of the sponge body 12) not only within the area of the hollow 40 but also outside the hollow 40 in the region between the liquid impermeable layer 16 and the perimetrical edge of the hollow 40.

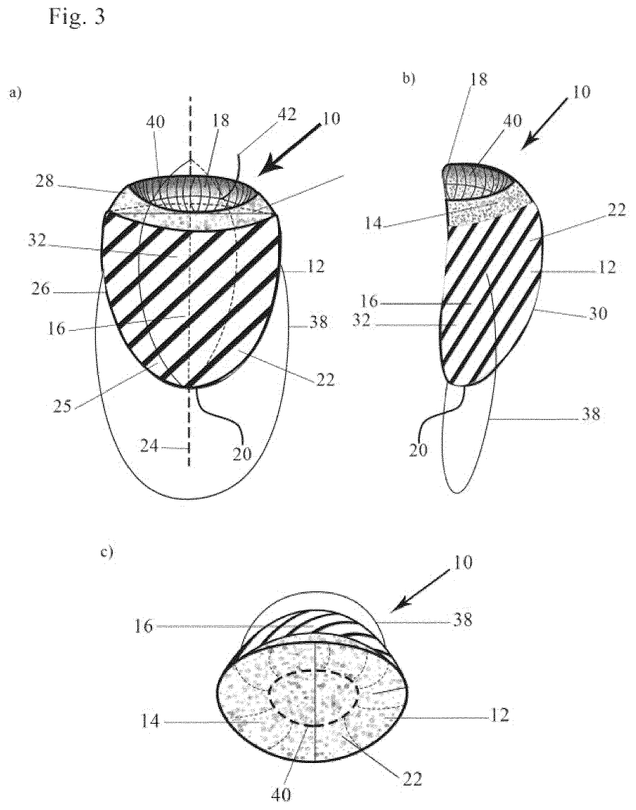

The hollow 40 is designed for cervix and easier fluid accumulation, and for faster fluid uptake to prevent blood stagnation at the cervix. The surface 42 comprises a portion of the material of the sponge structure 14 of the sponge body 12 and is generally concave in shape, such as in the form of a scoop, a hollow half-sphere, an oval, or any other form. Although the absorption surface 42 is said to be generally concavely shaped, it is to be understood that one or more sub-portions of the absorption surface 42 forming a part of the entire area of the absorption surface 42 may nevertheless have a plane or even convex shape. In other words, the absorption surface 42, although in general forming a concave depression or pocket, may be comprised of concave and non-concave surface portions. The hollow 40 may be formed on the main face 32 at a longitudinal position closer to the insertion end 18 than to the withdrawal end 20 (as shown exemplarily in FIGS. 2a-c and FIGS. 5a, 5b), or may be formed in the region of the insertion end 18 (as shown exemplarily in FIGS. 3a-c and 4a-c).

The sponge body 12 comprises at least a non-deformed state when it is fully expanded, a deformed state when it is compressed to occupy the smallest possible volume, and a partially deformed state in which the sponge body 12 fills the entire vaginal cavity by partially deforming the vaginal cavity walls at the contact site. In this way, the volume of fluids absorbed by the sponge body 12 is increased, while ensuring convenient vaginal adaptation. Reversible deformations of the sponge body 12 ensure that the adaptation to the vagina will take place continuously, while the user is both at rest and during movement. When the forces from the vaginal walls stop working on the sponge body 12, the deformed sponge body 12 recovers its original or partially original shape.

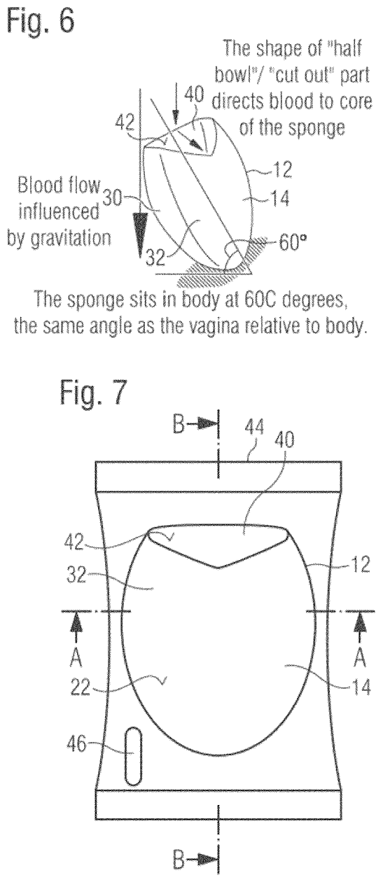

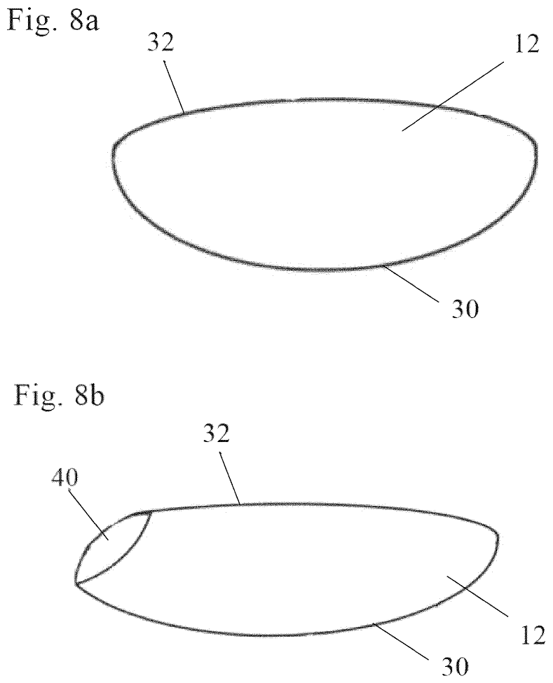

As can be seen from FIGS. 6, 7 and 8b, the absorption surface 42 defined by the hollow 40 has a concavely curved shape both in the width direction and the height direction of the sponge body 12 (the width and height, or thickness, dimensions are in a plane perpendicular to the longitudinal direction 24). On the side of the main face 32, the hollow 40 forms a deeper neck than on the side of the main face 30. The hollow 40 thus cuts out a relatively larger portion from the main face 32 than from the main face 30.

In certain embodiments, the sponge body 12 substantially maintains its shape and volume in a non-deformed (i.e. quiescent) state over a broad range of levels of humidification of the material of the sponge structure. It can be envisaged at least for some embodiments that in order to be able to store the sponge 10 for extended periods of time without degradation, even when packaged in a hermetic manner in a package, the material of the sponge structure 14 should rather be relatively dryer than moister. Certain embodiments therefore include in package a capsule or other suitable container (e.g., a sachet) together with the sponge 10, the capsule or container containing a predetermined amount of water or another liquid substance to be used for moisturizing the sponge body 12 before use.

Embodiments of the present invention also comprise a waterproof package 44 (FIG. 7) for said sponge 10. The package 44 is designed as a pouch in the example case shown in FIG. 7 and is configured to retain sterility and moisture of the body 12 of the sponge 10. It is to be understood that many different configurations of the pouch are conceivable and likewise many other designs of the waterproof package 44 than a pouch are conceivable, so long as the package 44 can retain a moist and sterile environment for the tampon 10. The sponge body 12 of the embodiment shown in FIGS. 6 and 7 has only the sponge structure 14, but no liquid impermeable layer 16. Also included in the package 44 is a schematically depicted capsule 46 containing an amount of, e.g., water which that the user can use to moisturize the sponge body 12 prior to its insertion into a human vagina.

The present invention also relates to a sponge as disclosed herein or a liquid composition as disclosed herein for inhibiting the growth of one or more pathogen(s) associated with a disorder or disease of the vaginal canal. Disorders or diseases of the vaginal canal are caused by one or more pathogen(s) such as bacteria and fungi. Disorder or diseases include but are not limited to dysbiosis, bacterial vaginosis, toxic shock syndrome and vulvovaginal candidiasis.

For example, the sponge for the vaginal canal as disclosed herein is used for preventing or treating vaginal dysbiosis. The liquid composition suitable for the vaginal canal as disclosed herein is used for preventing or treating dysbiosis. Dysbiosis (also called dysbacteriosis) is characterized as a disruption to the microbiota homeostasis caused by an imbalance in the microflora, changes in their functional composition and metabolic activities, or a shift in their local distribution. Such dysbiosis may lead to vulvovaginal candidiasis, bacterial vaginosis and toxic shock syndrome. The present invention also relates to a sponge for the vaginal canal as disclosed herein for use in preventing or treating vulvovaginal candidiasis. The present invention also relates to a liquid composition as disclosed herein for use in preventing or treating vulvovaginal candidiasis. The vulvovaginal candidiasis may be caused by Candida albicans, Candida glabrata, Candida kefyr, Candida krusei, Candida parapsilosis, and/or Candida tropicalis. For example, vulvovaginal candidiasis may be caused by Candida albicans. In some embodiments, said vulvovaginal candidiasis is prevented or treated in pregnant women. In some embodiments, said vulvovaginal candidiasis is prevented or treated in menstruating women. The present invention also relates to a sponge for the vaginal canal as disclosed herein for use in preventing or treating bacterial vaginosis. The present invention is also concerned with a liquid composition as disclosed herein for use in preventing or treating bacterial vaginosis. Bacterial vaginosis may be caused by Gardnerella vaginalis, Prevotella, Peptostreptococcus, Bacteroides spp., Leptotrichia spp, and/or Sneathia spp. In some embodiments, said bacterial vaginosis is prevented or treated in pregnant women. In some embodiments, said bacterial vaginosis is prevented or treated in menstruating women. The present invention also relates to a sponge for the vaginal canal or a liquid composition as disclosed herein for use in preventing toxic shock syndrome. Toxic shock syndrome may be caused by S. aureus. In some embodiments, said toxic shock syndrome is prevented in pregnant women. In some embodiments, said toxic shock syndrome is prevented in menstruating women.

In certain embodiments of uses as disclosed herein, the liquid composition inhibits the growth of pathogens, such as S. aureus, Candida spp, and/or Gardnerella vaginalis. In some embodiments, the liquid composition selectively inhibits the growth of one or more pathogen(s). In these embodiments the growth of one or more pathogen(s) is strongly inhibited such as at least 10-fold, at least 100-fold or at least 1000-fold. In certain embodiments, the liquid composition exhibits at least a 10-fold greater inhibition on one or more pathogen(s) than on L. gasseri and/or L. crispatus over a period of 8 hours. This is corroborated by Examples 3 and 4 of the enclosed invention. In particular, the liquid composition may exhibit at least a 10-fold greater inhibition on Gardnerella vaginalis than on L. gasseri and/or L. crispatus over a period of 8 hours. For example, said inhibition may be at least 50-fold, at least 60-fold at least 70-fold at least 80-fold, at least 100-fold, at least 500-fold or at least 1000-fold.

The sponge for the vaginal canal and/or the liquid composition according to the present invention advantageously protect the vaginal microflora and prevents pathogenic infection in particular during menstruation. The combination of the porous sponge structure impregnated with a liquid composition comprising one or more bacteriocin(s), as further defined in the claims, is advantageous as it preserves the vaginal natural defence mechanisms and thus strengthens the natural defence during menstruation. As demonstrated in Examples 1 and 2, the tampon according to the present invention selectively inhibits the growth of S. aureus, Candida albicans, and/or Gardnerella vaginalis. Hence, the liquid composition prevents pathogen growth and thus prevents e.g. vulvovaginal candidiasis and/or toxic shock syndrome. In doing so, the sponge according to the present invention may also be in use for a longer amount of time than a conventional tampon. The sponge according to the present invention may also be in use for a longer amount of time than a sponge not comprising a liquid composition comprising one or more bacteriocin(s), due to the inhibitory effect on pathogens. For example, the sponge according to the present invention may be used for up to 12 hours.

Although many features and advantages have been described above, along with the structural details and features of the invention, the description is provided as an exemplary embodiment of the invention. Changes may be made to the details, in particular in the form, size and arrangement of the materials, without departing from the principles of the invention, in accordance with the most widely understood terms used in the claims.

The invention may be further described by the following embodiments:
1. A sponge for a vaginal canal comprising:
a sponge body having a porous structure, the porous sponge structure forming at least part of a sponge outer surface,
wherein the sponge body is impregnated with a liquid composition comprising one or more bacteriocins.
2. The sponge of embodiment 1, wherein the porous sponge structure is made of a polysaccharide-based material comprising glucomannan.
3. The sponge of embodiments 1 or 2, wherein the sponge does not comprise Lactobacilli, in particular wherein the sponge does not comprise bacteria.
4. The sponge of any one preceding embodiment, wherein the liquid composition comprises a fixed amount of said one or more bacteriocin(s).
5. The sponge of any one preceding embodiment, wherein the sponge is moist.
6. The sponge of any one preceding embodiment, wherein said one or more bacteriocins is/are synthesised, wherein said one or more bacteriocins is/are synthesized, for example, in a bacterial strain.
4. The sponge of any one preceding embodiment, wherein the polysaccharide-based material is glucomannan.
5. The sponge of any one preceding embodiments, wherein the sponge is a tampon.
6. The sponge of any one preceding embodiment, wherein the sponge body impregnated with the composition promotes the vaginal microflora during menstruation, during follicular phase, during luteal phase and/or during pregnancy.
7. The sponge of any one preceding embodiment, wherein the sponge body impregnated with the composition promotes the vaginal microflora, in particular by inhibiting the growth of pathogens, especially S. aureus, Candida spp, and/or Gardnerella vaginalis, more in particular wherein the composition promotes the vaginal microflora by selectively inhibiting the growth of pathogens, and even more in particular wherein the composition promotes the vaginal microflora by selectively not inhibiting the growth of Lactobacilli.
8. The sponge of embodiment 7, wherein the sponge body impregnated with the composition inhibits the growth of S. aureus compared to medium alone under the same conditions, wherein the growth is inhibited, for example, by at least 20-fold or at least 50-fold or at least 100-fold or at least 200-fold or at least 350; tested after 8 h by the tampon sac method.
9. The sponge of embodiment 7 or 8, wherein the sponge body impregnated with the composition inhibits the growth of Candida spp. compared to medium alone under the same conditions, wherein the growth is inhibited, for example, by at least 30% or at least 40%, or at least 50% or at least 60% or at least 70% or at least 75%; tested after 8 h and 85% after 48 h by the tampon sac method.
10. The sponge of any one of embodiments 7 to 9, wherein the porous sponge structure impregnated with the composition inhibits the growth of Gardnerella vaginalis compared to medium alone under the same conditions, wherein the growth is inhibited, for example, by at least 50-fold at least 100-fold, or at least 250-fold, or at least 500-fold, or at least 1000-fold, or at least 5000-fold, or at least 10000-fold, or at least 20000-fold; tested after 24 h by the tampon sac method.
11. The sponge of any one preceding embodiment, wherein the sponge body impregnated with the composition inhibits the growth of Lactobacilli spp. compared to medium alone under the same conditions by at most 12-fold; tested after 8 h by the tampon sac method.
12. The sponge of any one preceding embodiment, wherein said one or more bacteriocins is/are selected from Class I, Class II, Class II, and Class IV bacteriocins from Gram-positive bacteria, wherein said one or more bacteriocins is/are, for example, selected from Class I and/or Class II bacteriocin from Gram-positive bacteria.
13. The sponge of any one preceding embodiment, wherein said one or more bacteriocins is/are a lantibiotic, wherein said one or more bacteriocins is/are, for example, a Type A or B lantibiotic.
14. The sponge of any one preceding embodiment, wherein said one or more bacteriocins is/are a Class II bacteriocin, wherein said Class II bacteriocin is, for example, a one-peptide bacteriocin or a two-peptide bacteriocin.
15. The sponge of any one preceding embodiment, wherein said one or more bacteriocins is/are a Class IIa bacteriocin, wherein the Class IIa bacteriocin is, for example, a circular bacteriocin.
16. The sponge of any one preceding embodiment, wherein the composition further comprises lactic acid, wherein the composition comprises lactic acid, for example, in an amount ensuring a pH value of said composition from 3.5 to 5.5, or from 3.7 to 5, or from 3.9 to 4.5, or around 4.2.
17. The sponge of any one preceding embodiment, wherein the liquid composition comprises an effective amount of one or more bacteriocin(s), in particular wherein the effective amount inhibits the growth of Gardnerella vaginalis, S. aureus, and/or Candida spp., and more in particular wherein the effective amount selectively inhibits the growth of Gardnerella vaginalis, S. aureus, and/or Candida spp.
18. The sponge of any one preceding embodiment, wherein the liquid composition comprises one bacteriocin.
19. The sponge of any one preceding embodiment, wherein said one or more bacteriocin(s) comprise(s) nisin.
20. The sponge of embodiment 19, wherein nisin is nisin A and/or nisin Z.
21. The sponge of any one preceding embodiment, wherein the liquid composition comprises one bacteriocin being nisin, in particular nisin A or nisin Z.
22. A method of manufacturing a sponge, the method comprising the steps of: providing a sponge body having a porous sponge structure, the porous sponge structure forming at least part of a sponge outer surface; and impregnating said porous sponge structure with a liquid composition comprising one or more bacteriocins to obtain the sponge according to any one of embodiments 1 to 21.
23. A liquid composition suitable for the vaginal canal comprising one or more bacteriocin(s) in an amount which promotes the vaginal microflora by inhibiting the growth of one or more pathogen(s).
24. The liquid composition of embodiment 23, wherein the composition has a pH value from 3.5 to 5.5.
25. The liquid composition of embodiment 23 or 24, wherein the composition does not comprise Lactobacilli, in particular wherein the composition does not comprise bacteria.
26. The liquid composition of any one of embodiments 23 to 25, wherein the one or more bacteriocin(s) comprise(s) nisin.
27. The liquid composition of any one of embodiments 23 to 26, wherein the liquid composition is an impregnant of a porous sponge.
28. The liquid composition of any one of embodiments 23 to 27, wherein the liquid composition comprises a fixed amount of the one or more bacteriocin(s).
29. The liquid composition of any one of embodiments 23 to 28, wherein the liquid composition comprises one bacteriocin.
30. The liquid composition of any one of embodiments 23 to 29, wherein said one or more bacteriocin(s) comprise(s) nisin.
31. The liquid composition of embodiments 30, wherein nisin is nisin A and/or nisin Z.
32. The liquid composition of any one of embodiments 23 to 31, wherein the liquid composition comprises one bacteriocin being nisin, in particular nisin A or nisin Z.
33. The liquid composition of any one of embodiments 23 to 32, wherein the one or more pathogens is selected from S. aureus, Candida spp, Gardnerella vaginalis and combinations thereof.
34. The liquid composition of any one of embodiments 23 to 33, wherein the one or more bacteriocin(s) are present at a total concentration of the liquid composition from 0.0001% (v/v) to 0.005% (v/v), 0.0002% (v/v) to 0.002% (v/v), 0.0004% (v/v) to 0.001% (v/v), 0.0005% (v/v) to 0.0014% (v/v), 0.0001% (v/v) to 0.0005% (v/v), or 0.0014% (v/v) to 0.008% (v/v), in particular wherein the one or more bacteriocin(s) are present at a total concentration of the liquid composition from 0.0006% (v/v) to 0.0013, 0.0007% (v/v) to 0.0012%, 0.0008% (v/v) to 0.0011%, or 0.0009% (v/v) to 0.0010%.
35. The liquid composition of any one of embodiments 23 to 34, wherein the liquid composition selectively inhibits the growth of one or more pathogen(s).
36. The liquid composition of any one of embodiments 23 to 35, wherein the liquid composition exhibits at least a 10-fold greater inhibition on one or more pathogen(s) than on L. gasseri and/or L. crispatus over a period of 8 hours, in particular wherein the liquid composition exhibits at least a 10-fold greater inhibition on Gardnerella vaginalis than on L. gasseri and/or L. crispatus over a period of 8 hours, more in particular at least 50-fold, and even more in particular at least 100-fold.
37. The liquid composition of any one of embodiments 23 to 36, wherein said one or more bacteriocins are further defined in any one of embodiments 12 to 15.
38. The liquid composition of any one of embodiments 23 to 37, wherein the composition is a gel, a solution, or a lubricant, in particular wherein the composition is a gel.
39. The liquid composition of any one of embodiments 23 to 38, wherein the liquid composition is a gel and wherein the gel further comprises a polysaccharide, in particular wherein the polysaccharide is glucomannan.
40. The liquid composition of any one of embodiments 23 to 39, wherein the composition further comprises lactic acid, wherein the composition comprises lactic acid, for example, in an amount ensuring a pH value of said composition from 3.5 to 5.5, or from 3.7 to 5, or from 3.9 to 4.5, or around 4.2.
41. The liquid composition of any one of embodiments 23 to 40, wherein the composition inhibits the growth of one or more pathogen(s) during menstruation, during follicular phase, during luteal phase and/or during pregnancy.
42. The sponge according to any one of embodiments 1 to 21 or the liquid composition according to any one of embodiment 23 to 41 for inhibiting the growth of one or more pathogen(s) associated with a disorder or disease of the vaginal canal.
43. The sponge according to any one of embodiments 1 to 21 or the liquid composition according to any one of embodiment 23 to 41 for use in preventing or treating dysbiosis.
44. The sponge according to any one of embodiments 1 to 21 or the liquid composition according to any one of embodiment 23 to 41 for use in preventing or treating vulvovaginal candidiasis.
45. The sponge according to any one of embodiments 1 to 21 or the liquid composition according to any one of embodiment 23 to 41 for use in preventing or treating bacterial vaginosis.
46. The sponge according to any one of embodiments 1 to 21 or the liquid composition according to any one of embodiment 23 to 41 for use in preventing toxic shock syndrome.

### EXAMPLES

The following examples are intended to illustrate the invention further, but are not limited to it. The examples describe technical features, and the invention also relates to combinations of the technical features presented in this section.

### Example 1—A Porous Sponge Impregnated with Bacteriocins Inhibits S. aureus and Candida albicans Growth

It was the objective of the present Example to determine the effect of the test substances on the growth of a yeast or bacterial culture. Specifically, the effect of various substances on the inhibition on pathogenic bacteria was tested in order to identify beneficial substances for impregnation of a porous sponge tampon.

### Method

The fungistatic effect of menstrual sponges on Candida albicans ATCC 10231 was evaluated. Furthermore, the antibacterial effect of menstrual sponges on S. aureus and Lactobacillus crispatus was evaluated. The tampon sac method as e.g. described in Nonfoux L, et al. (2018) [7] has been used. Briefly, samples of moist konjac sponges (glucomannan) (regular size with absorbency of 6-9 ml) were inserted into 532-ml sterile plastic bags in 10 mL brain heart infusion (BHI) broth for experiments with Candida spp. ATCC 10231 or Lactobacillus crispatus. The samples of moist konjac sponges (glucomannan) were inserted into 532-ml sterile plastic bags in 10 mL BHI broth infused with sheep's blood for experiments with S. aureus. (BHI and sheep's blood was mixed 1:1. The solution was inoculated with 10⁵ CFU/ml of Candida spp. ATCC 10231, Lactobacillus crispatus, S. aureus (e.g. S. aureus ST20140321). Excess air introduced during the insertion of the product into the bags was removed by manual deflation, and the bags were then hermetically sealed. One bag containing inoculated BHI broth without sponge was as used as positive control. The bags were incubated vertically at 37° C. shaking (around 200 rpm) for 8 h or 48 hours. Four ml of the solution was sampled from the plastic bag at the end of the incubation time. The tampons were kneaded in the bag for 5 seconds, and the liquid was squeezed out by compressing the tampon and collected for quantification of bacteria as described as follows. The number of yeast/bacteria in the solutions was estimated by standard spread plate technique using trypticase soy agar. Colonies were counted after 8 h, 24 h and 48 h of incubation at 30° C. in aerobic atmosphere.

### Results

Various different types of possible beneficial substances were tested in order to determine selective inhibition of pathogenic bacteria or yeast. Surprisingly, bacteriocins were identified to be exceptionally beneficial to inhibit pathogens, while not inhibiting Lactobacilli. Natamycin is a known antifungal chemical compound/medication used to treat fungal infection e.g. around the eye. Hence, it would be expected that natamycin would also show a fungistatic effect in the tampon sac method. When testing the inhibition of Candida albicans with natamycin from Streptomyces species by the tampon sac method, natamycin did not show an inhibitory effect (data not shown).

Similarly, it has been reported that polylysine has an antimicrobial effect against yeast, fungi and bacteria. When tested using the tampon sac methods, said antimicrobial effect against S. aureus could however not be detected. Specifically, the growth of S. aureus was induced from 1.0×10⁷ to 2.3×10⁸ and not inhibited at all. Hence, even though polylysine was expected to have an inhibitory effect on S. aureus, polylysine did not inhibit the growth of S. aureus.

In contrast, bacteriocins reliably inhibited the growth of pathogens. Specifically, the impregnation of the tampon with a liquid composition comprising nisin and lactic acid at a pH value of around 4.2 was tested. Said composition reliably inhibited C. albicans for 8, 24 or 48 hours of incubation using the tampon sac method. Specifically, the growth of C. albicans was assessed in four individual cultures and it was reduced by 78%, 75%, 76% and 73% after 8 hours. Furthermore, the growth of C. albicans was reduced by 32%, 35%, 39% and 28%, after 24 hours, respectively. Moreover, the growth of C. albicans was reduced by 83%, 84% and 87% after 48 hours, respectively. In sum, a reduction by around 75% after 8 hours could be observed; a reduction by around 30% after 24 hours could be observed; a reduction by around 85% after 48 hours could be observed. Percentages indicate the number of live cells (colony count) compared to control cells grown without menstrual sponge. The viability of control cells is equivalent to 100%.

In addition, the bacteriocins also effectively inhibited S. aureus. The colony numbers after 8 hours were detected as follows:

Inhibition of S.aureus

| positive | | | | |
|---|---|---|---|---|
| control | Sample 1 | Sample 2 | Sample 3 | Sample 4 |
| 1.0 × 10⁷ | 2.3 × 10⁴ | 2.8 × 10⁴ | 2.9 × 10⁴ | 3.0 × 10⁴ |

On average, the composition inhibited S. aureus by 2.6 log₁₀.

In order to ensure that the composition does not inhibit vaginal Lactobacilli, the inhibition on Lactobacillus crispatus was also tested:

Effect on Lactobacillus crispatus

| positive | | | | |
|---|---|---|---|---|
| control | Sample 1 | Sample 2 | Sample 3 | Sample 4 |
| 2.3 × 10⁴ | 2.8 × 10³ | 3.0 × 10³ | 1.1 × 10³ | 1.5 × 10³ |

On average, the composition inhibited Lactobacillus crispatus only to a minor degree, i.e. by 10.95 fold, which corresponds to 1.04 log₁₀.

In sum, the tested bacteriocins strongly inhibited the growth of Candida albicans and S. aureus, while Lactobacilli were only inhibited to a minor degree. Therefore, impregnation of a tampon with a liquid composition comprising one or more bacteriocin effectively inhibited Candida albicans and S. aureus. In contrast, other tested substances, which were previously reported to have an antibacterial or antifungal effect, did not have this effect.

### Example 2—A Porous Sponge Impregnated with Bacteriocins does not Comprise Bacteria and Inhibits Gardnerella vaginalis Growth

It is the aim of the present Example to verify that the impregnated porous sponge does not comprise bacteria. Separately, this Example tests the inhibition of Gardnerella vaginalis growth.

### Materials and Methods

Bacterial strain: Gardnerella vaginalis ATCC 49145.

Liquid culture medium and incubation method: sterile BHI (Brain-heart infusion, Liofilchem, ref. 620008) solution supplemented with 1% D-glucose and 2% horse serum (Thermofisher Scientific, Oxoid, ref. SR0035). The inoculated medium was incubated in 50 ml conical centrifugal tubes (Thermofisher Scientific, Nunc) in vertical position at 37° C. without shaking. The tubes were gently swirled for several times during the incubation. This method is considered equivalent to the tampon sac method.

Solid culture medium: sterile Chocolate Agar with Vitox plates (Thermofisher Scientific, Oxoid, ref. PO5090A). Plates were incubated for 43 hours at 37° C. in 5% CO2 atmosphere provided by CO2 Gen 2.5 L (Thermofisher Scientific, Oxoid, ref. CD0025A).

a) The Porous Sponge Impregnated with Bacteriocins does not Comprise Bacteria

While other products on the market may comprise bacteriocins, which are produced by bacteria within said product, the present invention in concerned with a porous sponge impregnated with a liquid composition comprising bacteriocins. Ideally, said porous sponge is free of bacteria.

To verify that said porous sponge is free of bacteria, such as Lactobacilli, three porous sponges (moist konjac sponges made out of glucomannan) were incubated in growth medium and potential bacterial growth was tested by counting potential colony forming units (CFUs). The tested sponges were impregnated with a liquid composition comprising nisin and lactic acid at a pH value of around 4.2.

Specifically, three sponges were inserted into 50 ml sterile tubes separately. 10 ml of liquid culture medium was added to each tube. The control tube contained medium only. After the incubation at 37° C. for 24 hours, the sponges were compressed several times using sterile pipette tips. 200 μl of medium was removed from each tube and spread on the culture plate. The plates were assessed after the incubation. This method is considered equal to the tampon sac method.

It was found that all plates had no colonies of microorganisms. Thus, the tested sponges were not contaminated bacteriologically. It can thus be concluded that the tested porous sponges are free of bacteria.

b) A Porous Sponge Impregnated with Bacteriocins Inhibits Gardnerella vaginalis Growth

As Gardnerella vaginalis is the most common cause of bacterial vaginosis, the inventors also tested the inhibitory effect of the porous sponge on the inhibition of growth of Gardnerella vaginalis.

Two independent tests (A, B) were carried out. Each test compared the bacterial growth with and without the sponge. The porous sponge (moist konjac sponges made out of glucomannan impregnated with a liquid composition comprising nisin and lactic acid at a pH value of around 4.2) was inserted into the bottom of 50 ml conical centrifugal tube. G. vaginalis ATCC 49145 was recovered from frozen storage by streaking and incubating it on the plate, and suspended in liquid medium at the optical density of OD₆₀₀ 0.12 (tests A, B). 100-fold and 10-fold lower densities failed to provide adequate bacterial growth during pre-testing.

15 ml of the bacterial suspension was poured on the sponge, and 15 ml were poured into the control tube. Serology pipette was used to allow even wetting of the sponge. The tubes were either loosely screwed in 5% CO₂ atmosphere (A) or tightly screwed (B) and incubated at 37° C. for 24 hours (tests A, B). The incubation for 8 hours was not considered for evaluation due to known slow growth rate of G. vaginalis (Janulaitiene, 2018, Fig B in S2 File). After incubation for 24 hours, the sponge was compressed in the tube ten times using the barrel of sterile syringe. The OD₆₀₀ of the suspensions was measured. 0.5 ml of culture medium was removed from each tube and serial 10-fold dilutions of culture were made in sterile PBS up to the factor of 10⁻⁷. 100 μl of suspensions of relevant dilutions were plated in two replicates. The colonies were counted manually on the plates suitable for reading. The count was used to calculate the colony forming units per millilitre (CFU/ml) for undiluted liquid cultures. The factor of bacterial growth inhibition was calculated by dividing the CFU/ml of the culture without sponge by the CFU/ml of the sponge incubation culture.

### The Results:

### (A)

| Incubation | OD₆₀₀ | CFU/ml of | Factor of inhibition of |
|---|---|---|---|
| type | at 24 h | undiluted culture | bacterial growth |
| no sponge | 2.49 | 5.05 × 10⁸ | 2.6 × 10⁶ |
| with sponge | 0.16 | 1.95 × 10² | |

### (B)

| Incubation | OD₆₀₀ | CFU/ml of | Factor of inhibition of |
|---|---|---|---|
| type | at 24 h | undiluted culture | bacterial growth |
| no sponge | 1.93 | 7.1 × 10ª | 3.6 × 104 |
| with sponge | 0.12 | 2 × 10⁴ | |

In these tests, the OD₆₀₀ of the cultures without the sponges demonstrated adequate growth potential of the starter G. vaginalis suspension. The tubes with the sponges did exhibit significantly less bacterial growth as shown by OD₆₀₀. In conclusion, the growth of G. vaginalis ATCC 49145 was inhibited by factors of 3.6×10⁴ and 2.6×10⁶ by the porous sponges impregnated with a liquid composition comprising a bacteriocin.

### Example 3—A Solution Comprising a Bacteriocin has an Inhibitory Effect on Gardnerella vaginalis

It is the aim of the present example to test the inhibitory effect of a composition comprising a bacteriocin against Gardnerella vaginalis, the main cause of bacterial vaginosis.

### Materials and Methods

A liquid solution comprising nisin was tested in the present Example (also called “Avodes solution”). Additionally, said composition comprised lactic acid at a pH value of around 4.2. The bacterial strain used in this Example was Gardnerella vaginalis ATCC 49145. The culture medium was the liquid medium BHI (Brain-heart infusion) solution (Liofilchem) supplemented with horse serum (Thermofisher Scientific, Oxoid). As a solid medium sterile Chocolate Agar was used (Thermofisher Scientific, Oxoid). All experiments were carried out at 37° C. under anaerobic conditions, provided by CO₂ Gen 2.5 (Thermofisher Scientific, Oxoid) to resemble the conditions in the vaginal canal as much as possible.

10 ml liquid medium was mixed with 6 ml of Avodes solution in sterile glass tubes. Each tube was inoculated with a final 4-6×10⁵ CFU/ml concentration of G. vaginalis. As controls, (i) a sterile glass tube with 10 ml of liquid medium (negative control) and no bacteria, (ii) a sterile glass tube with 10 ml of liquid medium inoculated with a final 4-6×10⁵ CFU/ml concentration of G. vaginalis (positive control), and (iii) sterile 10 ml glass tube mixed with 6 ml of solution (solution control). After incubating for 8 hours and 24 hours, 100 μl medium was removed from each tube, diluted (if needed), and spread on the culture plate. The results were evaluated after 48 hours of incubation. The provided solution was sterile (the negative control plates had no colonies of microorganisms).

| | CFU/ml at 0 h | CFU/ml at 8 h | CFU/ml at 24 h |
|---|---|---|---|
| Without solution | 4.15 × 10⁵ | 2 × 10⁷ | 3 × 10⁹ |
| With solution | 4.78 × 10⁵ | 1.97 × 10⁵ | 9.42 × 10⁵ |
| The factor of inhibition | | 1.02 × 10² | 3.18 × 10⁴ |
| of bacterial growth | | | |

### Results

This experiment shows that the “Avodes solution” had a significant inhibitory effect on the bacterial growth of Gardnerella vaginalis despite the incubation for several hours at 37° C. in growth medium. While the number of CFUs increased by around 50 fold after 8 hours in the solution control, the number of CFUs decreased by around 2 fold after 8 hours in the “Avodes solution”. The effect is even more drastic after an incubation of 24 hours: While the number of CFUs increased by around 7230 fold after 24 hours in the solution control, the number of CFUs only increased by around 2 2 fold after 24 hours in the “Avodes solution”. The factor if inhibition of bacterial growth amounts to 1.02×10² after 8 hours and 3.18×10⁴ after 24 hours.

These are very promising results showing that the composition comprising a bacteriocin effectively inhibits the growth of Gardnerella vaginalis, which is the main cause of bacterial vaginosis. As Gardnerella vaginalis is often found in low numbers in the vaginal macrobiota, it is expected that a composition comprising a bacteriocin such as nisin effectively keeps the number of Gardnerella vaginalis bacteria low and/or even reduces the number of Gardnerella vaginalis bacteria. Importantly, this exemplary solution can strongly inhibit Gardnerella vaginalis while only very mildly inhibiting bacteria promoting the vaginal microbiota such as Lactobacillus crispatus or Lactobacillus gasseri (see also next example).

### Example 4—A Solution and a Gel Comprising a Bacteriocin have a Strongly Inhibitory Effect on Gardnerella vaginalis and Only a Mild Inhibitory Effect on Lactobacillus Spp

It is the aim of the present example to test the inhibitory effect of a solution and a gel comprising a bacteriocin on Gardnerella vaginalis and Lactobacillus spp. As Gardnerella vaginalis is the main cause of bacterial vaginosis a strong inhibitory effect would be advantageous. In contrast, Lactobacillus spp support the vaginal microbiota so that it is advantageous if Lactobacillus spp are not inhibited or only mildly inhibited. The potential selective effect of a solution and a gel was also tested.

### Materials and Methods

A liquid solution comprising nisin was tested in the present Example (also called “Avodes Solution”). Furthermore, a gel comprising nisin was tested in the present Example (also called “Avodes Gel”). The Avodes gel additionally comprised glucomannan. Additionally, both compositions (solution and gel) comprised lactic acid at a pH value of around 4.2. The bacterial strains used in this Example were Gardnerella vaginalis ATCC 49145, Lactobacillus crispatus DSM 20584, Lactobacillus gasseri DSM 20243. Culture conditions: BHI (Brain-heart infusion, Sigma-Aldrich) supplemented with 1% D-glucose and 2% horse serum (Thermofisher Scientific, Oxoid) was used to grow Gardnerella vaginalis. The inoculated medium was incubated in sterile 15 ml conical centrifugal tubes in vertical position at 37° C. without shaking. To determine the number of CFU, the plates were incubated on Chocolate Agar with Vitox plates (Thermofisher Scientific, Oxoid) with CO₂Gen™ Compact Sachets (Thermofisher Scientific, Oxoid). Lactobacillus crispatus and Lactobacillus gasseri were grown in MRS broth with Tween 80 (Biolife). The inoculated medium was incubated in sterile 15 ml conical centrifugal tubes in vertical position at 37° C. without shaking and grown on MRS agar with Tween 80 (Biolife) with CO₂Gen™ Compact Sachets (Thermofisher Scientific, Oxoid). 0.5 ml Avodes Solution and 0.5 ml Avodes Gel were separately diluted in with 1 ml MRS or 1 ml BHI culture medium in 24 well plates and incubated at 37° C. for 24 hours. The control contained the respective medium only. 200 μl of medium was removed from each well and spread on the culture plate. The plates were assessed after the incubation.

To evaluate effect on G. vaginalis inhibition, two independent tests (Test A, Test B) were carried out with Avodes Solution and one with Avodes Gel then compared with the control which contained the respective medium and sodium lactate and lactic acid to maintain 4.2 pH. 1 mL of Avodes Solution was transferred in 24 well plate and mixed with 1 mL G. vaginalis inoculum in BHI broth supplemented with 1% D-glucose and 2% horse serum to give a final concentration of 10⁵ CFU/ml. The plates were incubated in 37° C. for 8 and 24 hours with CO₂Gen™ Compact Sachets to maintain anaerobic conditions. After incubation, serial 10-fold dilutions of the culture in sterile PBS were made up to a factor of 10⁻⁹ on chocolate agar. The colonies were counted manually on the plates suitable for reading. The count was used to calculate the colony forming units per millilitre (CFU/ml) for undiluted liquid cultures.

### Results

G. vaginalis with Avodes solution

| | | Avodes | |
|---|---|---|---|
| | Control | solution | Inhibition |
| | average | average | average |
| 0 h | 1.00 × 10⁵ | 1.00 × 10⁵ | |
| 8 h | 5.32 × 10⁷ | 2.14 × 10⁷ | 2.49 × 10 |
| 24 h | 6.42 × 10⁷ | 5.32 × 10³ | 1.21 × 10⁴ |

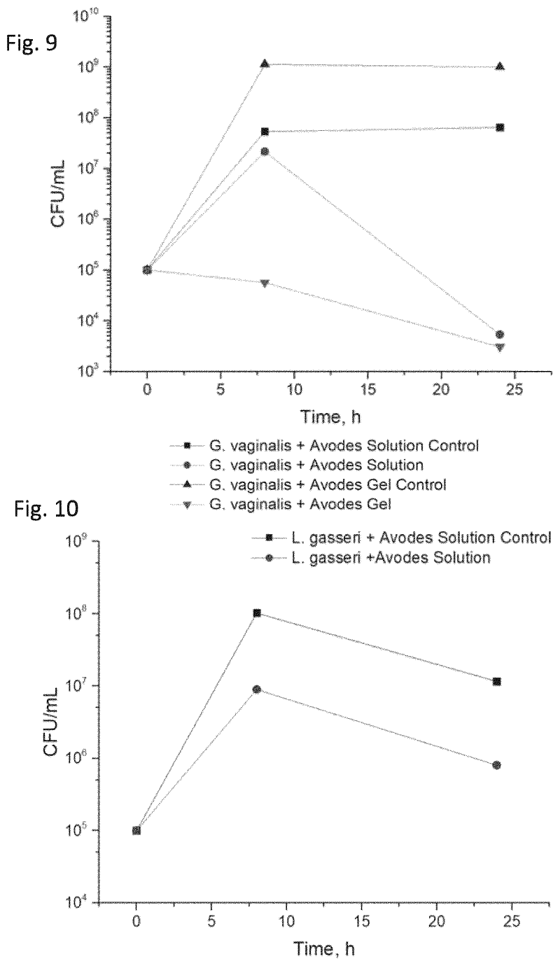

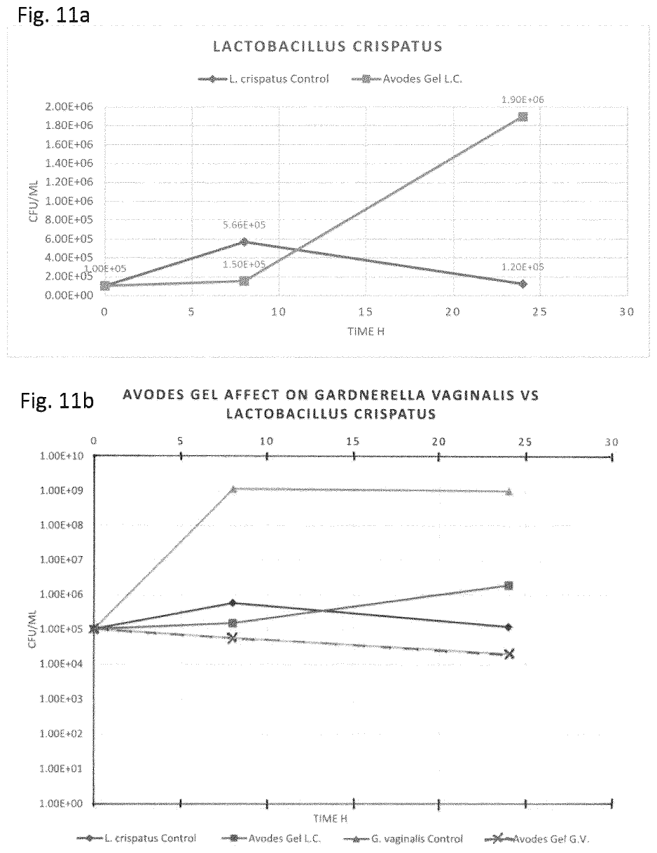

These results show that compositions (solution or gel) significantly inhibit the growth of Gardnerella vaginalis. Hence, these results further corroborate the data of the previous example. Additionally, this data demonstrates that the gel is even more advantageous as the inhibition effect is even larger amount to 2.01×10⁴ fold after 8 hours and 3.29×10⁵ fold after 24 hours. This shows that in particular a gel comprising nisin strongly inhibits the growth of Gardnerella vaginalis. The data reflected by the above Tables is also shown in FIG. 9.

G. vaginalis with Avodes Gel

| | Control | Avodes Gel | Inhibition |
|---|---|---|---|
| 0 h | 1.00 × 10⁵ | 1.00 × 10⁵ | |
| 8 h | 1.13 × 10⁹ | 5.64 × 10⁴ | 2.01 × 10⁴ |
| 24 h | 1.00 × 10⁹ | 2.01 × 10⁴ | 3.29 × 10⁵ |

The Evaluation of L. gasseri Growth Inhibition

To evaluate effect on L. gasseri inhibition, two independent tests (Test A, Test B) were carried out with Avodes Solution and one with Avodes Gel and then compared with the control which contains the respective medium and sodium lactate and lactic acid to maintain a pH of 4.2. 1 mL of Avodes Solution was transferred in 24 well plate and mixed with 1 mL L. gasseri inoculum in MRS broth acceding final concentration of 10⁵ CFU/ml. The plates were incubated in 37° C. for 8 and 24 hours with CO₂Gen™ Compact Sachets to maintain anaerobic conditions. After incubation, serial 10-fold dilutions of the culture in sterile PBS were made up to a factor of 10⁻⁹ on chocolate agar. The colonies were counted manually on the plates suitable for reading. The count was used to calculate the colony forming units per millilitre (CFU/ml) for undiluted liquid cultures.

### Results

L. gasseri with Avodes solution

| | | Avodes | |
|---|---|---|---|
| | Control | solution | Inhibition |
| | average | average | average |
| 0 h | 1.00 × 10⁵ | 1.00 × 10⁵ | |
| 8 h | 1.02 × 10⁸ | 8.95 × 10⁶ | 1.14 × 10¹ |
| 24 h | 1.15 × 10⁷ | 8.01 × 10⁵ | 1.44 × 10¹ |

This data shows that Lactobacilli such as L. gasseri are only very mildly inhibited by the Avodes solution. As e.g. L. gasseri is supporting the healthy microflora of the vaginal canal, it is particularly advantageous that the solution does only mildly inhibit Lactobacilli while very strongly inhibiting Gardnerella vaginalis. This selective inhibition is surprising and could not have been expected.

The Evaluation of L. crispatus Growth Inhibition

To evaluate effect on L. crispatus, two independent tests (Test A, Test B) were carried out with Avodes Solution and one with Avodes Gel then compared with the control which contains medium and sodium lactate and lactic acid to maintain 4.2 pH. 1 ml of Avodes Solution was transferred in 24 well plate and mixed with 1 mL L. gasseri inoculum in MRS broth gave a final concentration of 10⁵ CFU/ml. The plates were incubated in 37° C. for 8 and 24 hours with CO₂Gen™ Compact Sachets to maintain anaerobic conditions. After incubation, serial 10-fold dilutions of the culture in sterile PBS were made up to a factor of 10⁻⁹ on chocolate agar. The colonies were counted manually on the plates suitable for reading. The count was used to calculate the colony forming units per millilitre (CFU/ml) for undiluted liquid cultures.

### Results

L. crispatus with Avodes Solution

| | Test A | | | Test B | | | | | |
|---|---|---|---|---|---|---|---|---|---|
| | | Avodes | | | Avodes | | Control | Test | Inhibition |
| | Control | solution | Inhibition | Control | solution | Inhibition | average | average | average |
| 0 h | 1.00 × 10⁵ | 1.00 × 10⁵ | — | 1.00 × 10⁵ | 1.00 × 10⁵ | — | 1.00 × 10⁵ | 1.00 × 10⁵ | |
| 8 h | 8.77 × 10³ | 1.88 × 10² | 4.68 × 10¹ | 3.50 × 10⁶ | 1.58 × 10⁵ | 2.22 × 10¹ | 1.75 × 10⁶ | 7.91 × 10⁴ | 2.22 × 10¹ |
| 24 h | 1.32 × 10⁵ | 2.29 × 10² | 5.74 × 10² | 3.33 × 10⁶ | 1.71 × 10⁵ | 1.95 × 10¹ | 1.73 × 10⁶ | 8.56 × 10⁴ | 2.02 × 10¹ |

L. crispatus with Avodes Gel

| | Control | Avodes Gel | Inhibition |
|---|---|---|---|
| 0 h | 1.00 × 10⁵ | 1.00 × 10⁵ | |
| 8 h | 5.66 × 10⁵ | 1.50 × 10⁵ | 3.77 × 10⁰ |
| 24 h | 1.20 × 10⁵ | 1.90 × 10⁶ | 6.33 × 10⁻² |

This data further corroborates that the not only L. gasseri but also L. crispatus is only very mildly inhibited by the composition comprising a bacteriocin or is not inhibited at all. In particular, the gel did not at all or only very mildly inhibit both Lactobacilli tested. In sum, the data demonstrates that the composition (solution or gel) selectively inhibits bacteria: The composition strongly inhibits Gardnerella vaginalis and only very mildly inhibits Lactobacilli. Additionally, the gel even enhanced the growth of L. crispatus by at least one log unit, i.e. at least 10-fold. This selectively inhibition is expected to support the natural microflora of the vaginal canal. Due to the strong effect, this selectively inhibition is also expected to prevent bacterial vaginosis, candidiasis and/or dysbiosis. Due to the strong effect, this selectively inhibition is also expected to treat bacterial vaginosis, candidiasis and/or dysbiosis.

### REFERENCES

1. J. Yano, J. D. Sobel et al. Current patient perspectives of vulvovaginal candidiasis: incidence, symptoms, management and posttreatment outcomes. 2019 Mar. 29; 19 (1): 48. doi: 10.1186/s12905-019-0748-8.
2. F. Bernardis et al. Candida vaginitis: virulence, host response and vaccine prospects. Med Mycol. 2018 Apr. 1; 56 (suppl_1): 26-31.doi: 10.1093/mmy/myx139.
3. J. D. Sobel Vulvovaginal candidiasis: epidemiologic, diagnostic, and therapeutic considerations. Am J Obstet Gynecol. 1998 February; 178 (2): 203-11.doi: 10.1016/s0002-9378 (98) 80001-x.
4. E. Gillet et al. Bacterial vaginosis is associated with uterine cervical human papillomavirus infection: a meta-analysis. BMC Infect Dis. 2011; 11:10. doi: 10.1186/1471-2334-11-10, https://www.ncbi.nlm.nih.gov/pmc/articles/PMC3023697/5.
5. F. M. T. Lewis et al. Vaginal Microbiome and Its Relationship to Behavior, Sexual Health, and Sexually Transmitted Diseases. Obstet Gynecol. Author manuscript; available in PMC 2019 Sep. 13. doi: 10.1097/AOG.0000000000001932
6. K. Peebles et al. High Global Burden and Costs of Bacterial Vaginosis: A Systematic Review and Meta-Analysis. Sex Transm Dis. 2019 May; 46 (5): 304-311. doi: 10.1097/OLQ.0000000000000972.
7. J. D. Sobel Bacterial vaginosis. Annu Rev Med. 2000; 51:349-56.doi: 10.1146/annurev.med.51.1.349.
8. M. Petrova et al. Lactobacillus species as biomarkers and agents that can promote various aspects of vaginal health. Front Physiol. 2015; 6:81.doi: 10.3389/fphys.2015.00081
9. J. Kaewsrichan et al. Selection and identification of anaerobic lactobacilli producing inhibitory compounds against vaginal pathogens. FEMS Immunol Med Microbiol. 2006 October; 48 (1): 75-83.doi: 10.1111/j.1574-695X.2006.00124.x.
10. J. Garza et al. Cytokine profiles and Lactobacillus species presence in prie-menopausal subjects with genital Mycoplasma genitalium or Ureaplasma urealyticum colonization. Womens Health (Lond). 2021; 17:17455065211009181. doi: 10.1177/17455065211009181
11. W. Mendling. Vaginal Microbiota Adv Exp Med Biol. 2016; 902:83-93. doi: 10.1007/978-3-319-31248-4_6.
12. A. Kiecka. Can Lactobacillus spp. be a Factor Reducing the Risk of Miscarriage? Pol J Microbiol. 2021 December; 70 (4): 431-446; Eckert L O et. al. Relationship of vaginal bacteria and inflammation with conception and early pregnancy loss following in vitro fertilization. Infect Dis Obstet Gynecol. (2003) 11:11-7.
13. Amabebe E, Anumba D O C. The Vaginal Microenvironment: The Physiologic Role of Lactobacilli. Front Med (Lausanne). 2018 Jun. 13; 5:181
14. D. Eschenbach et al. Influence of the Normal Menstrual Cycle on Vaginal Tissue, Discharge, and Microflora. Clinical Infectious Diseases, Volume 30, Issue 6, June 2000, Pages 901-907
15. S. Srinivasan et al. Temporal Variability of Human Vaginal Bacteria and Relationship with Bacterial Vaginosis. PLOS One. 2010; 5 (4): e10197
16. C. Bradshaw, J. D. Sobel Current Treatment of Bacterial Vaginosis-Limitations and Need for Innovation. J Infect Dis. 2016 Aug. 15; 214 (Suppl 1): S14-S20. doi: 10.1093/infdis/jiw159
17. J. Sobel Recurrent vulvovaginal candidiasis. Am J Obstet Gynecol. 2016 January; 214 (1): 15-21. doi: 10.1016/j.ajog.2015.06.067. Epub 2015 Jul. 9.
18. Vostral, S L, 2011. Rely and Toxic Shock Syndrome: A Technological Health Crisis Yale J. Biol. Med. 84, 447-459
19. Nicole, W., 2014. A Question for Women's Health: Chemicals in Feminine Lubricants, Environmental Health Perspect 122, A70-75
20. Nonfoux L, et al. Impact of Currently Marketed Tampons and Menstrual Cups on Staphylococcus aureus Growth and Toxic Shock Syndrome Toxin 1 Production In Vitro. Appl Environ Microbiol. 2018 May 31; 84 (12): e00351-18
21. S. Srinivasan et al. Temporal Variability of Human Vaginal Bacteria and Relationship with Bacterial Vaginosis. PLOS One. 2010; 5 (4): e10197
22. Janulaitiene M, Gegzna V, Baranauskiene L, Bulavaitė A, Simanavicius M, Pleckaityte M. Phenotypic characterization of Gardnerella vaginalis subgroups suggests differences in their virulence potential. PLOS One. 2018 Jul. 12; 13 (7): e0200625. doi: 10.1371/journal.pone.0200625. eCollection 2018.
23. J. Sobel et al. Conventional oral and secondary high dose vaginal metroni-dazole therapy for recurrent bacterial vaginosis: clinical outcomes, im-pacts of sex and menses. Infect Drug Resist. 2019; 12:2297-2307. Published online 2019 Jul. 24.

1. A sponge for a vaginal canal comprising:
a sponge body having a porous structure, the porous sponge structure forming at least part of a sponge outer surface,
wherein the porous sponge structure is made of a polysaccharide-based material comprising glucomannan,
wherein the sponge does not comprise bacteria, and
wherein the sponge body is impregnated with a liquid composition comprising one or more bacteriocin(s).

2. The sponge of claim 1, wherein liquid composition comprises a fixed amount of said one or more bacteriocin(s).

3. The sponge of claim 1, wherein the sponge is moist and/or is a tampon.

4. The sponge of claim 1, wherein the polysaccharide-based material is glucomannan.

5. (canceled)

6. The sponge of claim 1, wherein the sponge body impregnated with the composition promotes the vaginal microflora during menstruation, during follicular phase, during luteal phase and/or during pregnancy.

7. The sponge of claim 1, wherein the sponge body impregnated with the composition promotes the vaginal microflora by inhibiting the growth of pathogens, in particular S. aureus, Candida spp, and/or Gardnerella vaginalis.

8. (canceled)

9. (canceled)

10. (canceled)

11. The sponge of claim 1, wherein said one or more bacteriocins is/are selected from Class I, Class II, Class II, and Class IV bacteriocins from Gram-positive bacteria.

12. (canceled)

13. The sponge of claim 1, wherein said one or more bacteriocin(s) comprise(s) nisin, optionally wherein nisin is nisin A and/or nisin Z.

14. (canceled)

15. (canceled)

16. The sponge of claim 1, wherein the composition further comprises lactic acid, optionally wherein the composition comprises lactic acid in an amount ensuring a pH value of said composition from 3.5 to 5.5, or from 3.7 to 5, or from 3.9 to 4.5, or around 4.2.

17. A method of manufacturing a sponge, the method comprising the steps of:
providing a sponge body having a porous sponge structure, the porous sponge structure forming at least part of a sponge outer surface; and
impregnating said porous sponge structure with a liquid composition comprising one or more bacteriocins to obtain the sponge according to claim 1.

18. A liquid composition suitable for the vaginal canal comprising one or more bacteriocin(s) in an amount which promotes the vaginal microflora by inhibiting the growth of one or more pathogen(s),
wherein the composition has a pH value from 3.5 to 5.5,
wherein the composition does not comprise bacteria, and
wherein the one or more bacteriocin(s) comprise(s) nisin.

19. The liquid composition of claim 18, wherein the composition is an impregnant of a porous sponge and/or wherein the composition comprises a fixed amount of the one or more bacteriocin(s).

20. (canceled)

21. The liquid composition of claim 18, wherein the liquid composition comprises one bacteriocin, optionally wherein the one bacteriocin is nisin, in particular nisin A or nisin Z.

22. (canceled)

23. The liquid composition of claim 18, wherein the one or more pathogens is selected from S. aureus, Candida spp, Gardnerella vaginalis and combinations thereof.

24. The liquid composition of claim 18, wherein the one or more bacteriocin(s) are present at a total concentration of the liquid composition from 0.0001% (v/v) to 0.005% (v/v), 0.0002% (v/v) to 0.002% (v/v), 0.0004% (v/v) to 0.001% (v/v), 0.0005% (v/v) to 0.0014% (v/v), 0.0001% (v/v) to 0.0005% (v/v), or 0.0014% (v/v) to 0.008% (v/v).

25. The liquid composition of claim 18, wherein the liquid composition is a gel, a solution or a lubricant, in particular wherein the liquid composition is a gel further comprising glucomannan.

26. (canceled)

27. The liquid composition of claim 18, wherein the composition further comprises lactic acid.

28. The liquid composition of claim 18, wherein the composition inhibits the growth of one or more pathogen(s) during menstruation, during follicular phase, during luteal phase and/or during pregnancy.

29. (canceled)

30. (canceled)

31. (canceled)

32. (canceled)

33. A method of treating or preventing dysbiosis, vulvovaginal candidiasis and/or bacterial vaginosis, or of preventing toxic shock syndrome comprising applying a sponge for a vaginal canal comprising a sponge body having a porous structure, the porous sponge structure forming at least part of a sponge outer surface, wherein the porous sponge structure is made of a polysaccharide-based material comprising glucomannan, wherein the sponge does not comprise bacteria, and wherein the sponge body is impregnated with a liquid composition comprising one or more bacteriocin(s); or
a liquid composition suitable for the vaginal canal comprising one or more bacteriocin(s) in an amount which promotes the vaginal microflora by inhibiting the growth of one or more pathogen(s), wherein the composition has a pH value from 3.5 to 5.5, wherein the composition does not comprise bacteria, and wherein the one or more bacteriocin(s) comprise(s) nisin.
